(19) United States
(12) Patent Application Publication
(10) Pub. No.: US 2024/0192235 A1
(43) Pub. Date: Jun. 13, 2024

(54) CERAMIDE AND SPINGOMYELIN IN NEUROLOGICAL DISORDERS

(71) Applicants: Duke University, Durham, NC (US); METABOLOMICS LABORATORY, BAKER HEART & DIABETE INSTITUTE, Melbourne, Vitoria (AU); UNIVERSITY OF TEXAS HEALTH CENTER AT SAN ANTONIO, San Antonio, TX (US); INDIANA ALZHEIMER DISEASE CENTER AND DEPT. OF RADIOLOGY AND IMAGINING SCIENCES, Indianapolis, IN (US); SUNY DOWNSTATE MEDICAL CENTER, Brooklyn, NY (US); INTITUTE FOR SYSTEMS BIOLOGY, Seattle, WA (US); HELMHOLTZ ZENTRUM MUNCHEN-GERMAN RESEARCH CENTER FOR ENVIRONMENTAL HEALTH, Neuherberg (DE)

(72) Inventors: Rima F. Kaddurah-Daouk, Belmont, MA (US); Gregory Louie, Durham, NC (US); Matthias Arnold, Neuherberg (DE); Rebecca A. Baillie, Durham, NC (US); Xianlin Han, San Antonio, TX (US); Andrew J. Saykin, Bloomington, IN (US); Kwangsik Nho, Bloomington, IN (US); Herman W. Moreno, Brooklyn, NY (US); Cory Funk, Seattle, WA (US); Priyanka Baloni, Seattle, WA (US); Gabriele Kastenmueller, Neuherberg (DE); Peter Meikle, Melbourne (AU)

(21) Appl. No.: 18/554,852

(22) PCT Filed: Apr. 22, 2022
(86) PCT No.: PCT/US2022/025904

§ 371 Date: Oct. 11, 2023

Related U.S. Application Data
(60) Provisional application No. 63/178,978, filed on Apr. 23, 2021.

(51) Int. Cl. G01N 33/92; A61B 5/055; A61B 6/50; A61K 31/137; A61P 25/28; C12Q 1/6883; G01N 33/573; G16B 5/00; G16H 50/20

(52) U.S. Cl. CPC G01N 33/92; A61B 5/055; A61B 6/501; A61K 31/137; A61P 25/28; C12Q 1/6883; G01N 33/573; G16B 5/00; G16H 50/20; C12Q 2600/156; G01N 2405/08; G01N 2800/28

(57) ABSTRACT

Described herein are compositions and methods for assessing and modulating ceramide and sphingomyelin in neurological disorders.

### CROSS-REFERENCE TO RELATED APPLICATIONS

This application claims priority to U.S. Provisional Patent Application No. 63/178,978, filed on Apr. 23, 2021, which is incorporated by reference herein in its entirety.

### FEDERALLY SPONSORED RESEARCH

This invention was made with government support under grant numbers AG058942 and AG057452 awarded by the National Institutes of Health. The government has certain rights in the invention.

### TECHNICAL FIELD

Described herein are compositions and methods for assessing and modulating ceramide and sphingomyelin in neurological disorders.

### BACKGROUND

To date, approximately 400 trials of experimental Alzheimer's disease (AD) treatments have failed. In the wake of such large-scale failure, the amyloid hypothesis has been called into question and researchers are pursuing alternative approaches, with a greater focus on the complex mechanisms underlying this neurodegenerative disease. In an effort to address this knowledge gap, the NIH-funded Accelerating Medicines Partnership-Alzheimer's Disease (AMP-AD) has successfully generated new hypotheses and insights around AD by producing large, publicly available datasets. The knowledge gained from this initiative has caused a major paradigm shift in research focus, resulting in novel targets and testable hypotheses, which are currently being investigated in clinical phase 1 and 2 trials, aimed at neuroprotection and anti-neuroinflammation. These new hypotheses also open the door for drug repositioning and development.

While the central neuropathological features of AD are accumulation of misfolded β-amyloid (Aβ) plaques and phosphorylated tau proteins, brain atrophy and neuronal loss are equally important features. The relationship between Aβ accumulation, tau phosphorylation and neuronal loss are not clear. What is clear is that AD etiology is multifactorial, with genetic contributions, protein mis-trafficking and turnover, altered glucose metabolism and lipid metabolism failures. For example, recent studies have clarified the important relationship between the immune system and lipid metabolism, and more than half of the genes implicated in AD via genetic association screens are linked to lipid metabolism and inflammation. APOE4, the strongest genetic risk factor for late onset AD, is centrally involved in lipid metabolism, including the transport of cholesterol to neurons from astrocytes. Additionally, several independent genetic association studies with metabolite levels have reported replicable associations of the APOE gene locus with blood levels of sphingolipid species. Furthermore, lipids, including sphingomyelins (SMs), have been shown to be disrupted in AD. However, the impacts of lipid alterations on AD pathogenesis are not fully understood.

Thus, what is needed are methods for assessing and modulating the levels of novel biological targets, such as ceramide and sphingomyelin, in neurological disorders such as AD, Parkinson's Disease, and multiple sclerosis.

### SUMMARY

One embodiment described herein is a method for the classification and treatment of a neurological disorder in a subject based on the subject's metabolic profile and genetic screening, the method comprising one or more of the following: identifying and stratifying subjects afflicted with a neurological disorder to subgroups based on their metabolic profiles, biomarker metabolites and ratios of biomarker metabolites that define unique metabolic conditions related to aberrations in ceramide and sphingomyelin anabolism, catabolism, or homeostasis and common identity among subgroups of subjects; evaluating the trajectory of disease within each stratified subgroup of subjects and their response to a therapeutic treatment; identifying defects in ceramide or sphingomyelin transport and/or biosynthesis of biomarker metabolites within a metabolic pathway or across metabolic pathways using ratios of biomarker metabolites to inform about changes in enzyme activities or transporters; and identifying genetic bases of ceramide and sphingomyelin metabolic profile characteristics or defects (SNPs/genetic variants in key enzymes and transporters) using metabolite genome-wide association study (mGWAS) analysis. In one aspect, the method further comprises one or more of the following: using combined metabotype and genotype data and genome-scale metabolic models to better stratify subjects with the neurological disorder and to inform about mechanisms and treatment selection; suggesting a therapeutic approach to correct metabolic defects in metabolic profile in stratified subgroups of subjects; and comparing and contrasting metabolic defects noted in inborn errors of metabolism that have the neurological disorder and using knowledge gained in treatment of inborn errors of metabolism to inform treatment for neurological disorder. In another aspect, the method further comprises administering to the subjects an effective amount of a therapy to prevent and/or treat the neurological disorder affected by one or more metabolic defects. In another aspect, the neurological disorder is a neuropsychiatric or neurodegenerative symptom associated with neurological diseases or cognitive impairment, including depression, anxiety, dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's disease, young-onset dementia, Korsakoff's syndrome, Huntington's disease, HIV-associated neurocognitive disorders, multiple sclerosis, or other cognitive impairment, neuropsychiatric, or neurodegenerative disorders.

Another embodiment described herein is a method for stratifying and treating a subject having a neurological disorder, or at risk of developing a neurological disorder, based on the subject's metabolic profile, the method comprising: analyzing a sample from a subject to determine concentration levels or ratios of one or more biomarker metabolites related to ceramide and sphingomyelin anabolism, catabolism, or homeostasis in the sample from the subject; determining if the subject has a metabolic defect related to disrupted ceramide and sphingomyelin anabolism, catabolism, or homeostasis, or if the subject's gut microbiome has a defect related to disrupted ceramide and sphingomyelin anabolism, catabolism, or homeostasis, or combinations thereof based on the measured concentration levels and calculated ratios of the one or more ceramide and sphingomyelin anabolism, catabolism, or homeostasis biomarker metabolites in the sample as compared to a control sample; stratifying the subject into a subgroup of subjects, wherein an individual subgroup of subjects is defined by a unique and specific ceramide and sphingomyelin anabolism, catabolism, or homeostasis profile based on the measured concentration levels and calculated ratios of the one or more biomarker metabolites in the sample as compared to a control sample and the biomarker metabolite defect determined for the subject. In one aspect, the method further comprises treating the neurological disorder by administering to the subgroup of subjects an effective amount of a therapy sufficient to attenuate, reduce, or eliminate the symptoms of neurological disorder, wherein the therapy is determined by the unique and specific metabolic profile of the subgroup of subjects. In another aspect, the one or more biomarker metabolites comprises one or more of: sphingomyelins (SM): SM (OH) C14:1 (SM C15:0); SM C16:0; SM (OH) C16:1 (SM C17:0); SM C16:1; SM C18:0; SM C18:1; SM C20:2; SM (OH) C22:1 (SM C23:0); SM (OH) C22:2 (SM C23:1); SM C24:0; SM (OH) C24:1 (SM C25:0); SM C24:1; SM C26:0; SM C26:1; SM (d43:1); SM (d34:1); SM (d32:0); SM (d38:3); SM (d33:0); SM (d38:2); SM (d33:1); SM (d32:1); SM (d44:1); SM (d42:1); SM (d41:1); SM (d35:1); SM (d36:1); SM (d44:2); SM (d34:2); SM (d36:2); or combinations thereof; ceramides (Cer): Cer (d18:1,14:0); Cer (d18:1,16:1); Cer (d18:0,16:1); Cer (d16:1,16:0); Cer (d18:2,16:0); Cer (d18:1,16:0); Cer (d18:0,16:0); Cer (d16:0,18:1); Cer (d16:1,18:0); Cer (d18:1,18:0); Cer (d18:1, 22:1); Cer (d18:0, 22:1); Cer (d18:2, 22:0); Cer (d18:1, 22:0); Cer (d18:1, 23:1); Cer (d18:0, 23:1); Cer (d18:2, 23:0); Cer (d18:1, 23:0); Cer (d18:1, 24:2); Cer (d18:0, 24:2); Cer (d16:1, 24:1); Cer (d18:2, 24:1); Cer (d18:1, 24:1); Cer (d18:0, 24:1); Cer (d16:1, 24:0); Cer (d18:2, 24:0); Cer (d18:1, 24:0); Cer (d18:0, 24:0); Cer (d20:1, 24:0); Cer (d20:0, 24:0); Cer (d18:1, 26:1); Cer (d18:0, 26:1); Cer (d18:2, 26:0); Cer (d18:1, 26:0); Cer (d18:0, 26:0); or combinations thereof; or sphingosine-1-phosphate (S1P). In another aspect, the one or more biomarker metabolites comprises S1P. In another aspect, the one or more biomarker metabolites related to ceramide or sphingomyelin metabolism comprises one or more sphingomyelins (SM): SM (OH) C14:1 (SM C15:0); SM C16:0; SM (OH) C16:1 (SM C17:0); SM C16:1; SM C18:0; SM C18:1; SM C20:2; SM (OH) C22:1 (SM C23:0); SM (OH) C22:2 (SM C23:1); SM C24:0; SM (OH) C24:1 (SM C25:0); SM C24:1; SM C26:0; SM C26:1; SM (d43:1); SM (d34:1); SM (d32:0); SM (d38:3); SM (d33:0); SM (d38:2); SM (d33:1); SM (d32:1); SM (d44:1); SM (d42:1); SM (d41:1); SM (d35:1); SM (d36:1); SM (d44:2); SM (d34:2); SM (d36:2); or combinations thereof; and wherein one or more ratios of the SM is calculated. In another aspect, the one or more biomarker metabolites related to ceramide or sphingomyelin metabolism comprises SM (d43:1) and SM (d34:1), and wherein a ratio of SM (d43:1)/SM (d34:1) is calculated. In another aspect, the subject is determined as having the neurological disorder or an increased risk of the neurological disorder when the sample comprises a higher ratio of SM (d43:1)/SM (d34:1) compared to the control sample. In another aspect, the one or more biomarker metabolites comprises one or more ceramides (Cer). In another aspect, the method further comprises: administering to the subject a therapeutically effective amount of one or more ceramides, sphingomyelins, and/or any pharmaceutically acceptable derivatives, esters, salts, solvates, hydrates, analogs, or prodrugs thereof; and/or administering to the subject a therapeutically effective amount of one or more therapeutic agents capable of modulating (increasing or decreasing) the concentration levels or ratios of one or more primary or sphingomyelins, activating the endogenous production of one or more ceramides or sphingomyelins, and/or decreasing the breakdown of one or more ceramides or sphingomyelins; and/or administering to the subject a therapeutically effective amount of a sphingosine-1-phosphate receptor (S1PR) modulator including one or more of fingolimod, SEW2871, siponimod, ozanimod, ceralifimod, GSK2018682, ponesimod, KRP203, cenerimod, amiselimod (MT-1303), etrasimod, laquinimod, derivatives thereof, or combinations thereof.

Another embodiment described herein is a method for detecting a neurological disorder in a subject, the method comprising: analyzing a sample from a subject and a control sample from a control subject or population of subjects with normal cognition; measuring the concentration levels or ratios of one or more biomarker metabolites related to ceramide or sphingomyelin metabolism in the sample from the subject and the control sample; and determining the subject as having a neurological disorder or an increased risk of a neurological disorder when the concentration levels or ratios of the one or more biomarker metabolites in the sample from the subject are different from (greater than or less than) the concentration levels or ratios of the one or more biomarker metabolites in the control sample. In one aspect, the one or more biomarker metabolites related to ceramide or sphingomyelin metabolism comprises one or more sphingolipids including: sphingomyelins (SM): SM (OH) C14:1 (SM C15:0); SM C16:0; SM (OH) C16:1 (SM C17:0); SM C16:1; SM C18:0; SM C18:1; SM C20:2; SM (OH) C22:1 (SM C23:0); SM (OH) C22:2 (SM C23:1); SM C24:0; SM (OH) C24:1 (SM C25:0); SM C24:1; SM C26:0; SM C26:1; SM (d43:1); SM (d34:1); SM (d32:0); SM (d38:3); SM (d33:0); SM (d38:2); SM (d33:1); SM (d32:1); SM (d44:1); SM (d42:1); SM (d41:1); SM (d35:1); SM (d36:1); SM (d44:2); SM (d34:2); SM (d36:2); or combinations thereof; ceramides (Cer): Cer (d18:1,14:0); Cer (d18:1,16:1); Cer (d18:0,16:1); Cer (d16:1,16:0); Cer (d18:2,16:0); Cer (d18:1,16:0); Cer (d18:0,16:0); Cer (d16:0,18:1); Cer (d16:1,18:0); Cer (d18:1,18:0); Cer (d18:1, 22:1); Cer (d18:0, 22:1); Cer (d18:2, 22:0); Cer (d18:1, 22:0); Cer (d18:1, 23:1); Cer (d18:0, 23:1); Cer (d18:2, 23:0); Cer (d18:1, 23:0); Cer (d18:1, 24:2); Cer (d18:0, 24:2); Cer (d16:1, 24:1); Cer (d18:2, 24:1); Cer (d18:1, 24:1); Cer (d18:0, 24:1); Cer (d16:1, 24:0); Cer (d18:2, 24:0); Cer (d18:1, 24:0); Cer (d18:0, 24:0); Cer (d20:1, 24:0); Cer (d20:0, 24:0); Cer (d18:1, 26:1); Cer (d18:0, 26:1); Cer (d18:2, 26:0); Cer (d18:1, 26:0); Cer (d18:0, 26:0); or combinations thereof; or sphingosine-1-phosphate (S1P). In another aspect, the one or more biomarker metabolites comprises S1P. In another aspect, the subject is determined as having the neurological disorder or an increased risk of the neurological disorder when the sample comprises lower concentration levels of S1P compared to the control sample. In another aspect, the one or more biomarker metabolites related to ceramide or sphingomyelin metabolism comprises one or more sphingomyelins (SM): SM (OH) C14:1 (SM C15:0); SM C16:0; SM (OH) C16:1 (SM C17:0); SM C16:1; SM C18:0; SM C18:1; SM C20:2; SM (OH) C22:1 (SM C23:0); SM (OH) C22:2 (SM C23:1); SM C24:0; SM (OH) C24:1 (SM C25:0); SM C24:1; SM C26:0; SM C26:1; SM (d43:1); SM (d34:1); SM (d32:0); SM (d38:3); SM (d33:0); SM (d38:2); SM (d33:1); SM (d32:1); SM (d44:1); SM (d42:1); SM (d41:1); SM (d35:1); SM (d36:1); SM (d44:2); SM (d34:2); SM (d36:2); or combinations thereof; and wherein one or more ratios of the SM is calculated. In another aspect, the one or more biomarker metabolites related to ceramide or sphingomyelin metabolism comprises SM (d43:1) and SM (d34:1), and wherein a ratio of SM (d43:1)/SM (d34:1) is calculated. In another aspect, the subject is determined as having the neurological disorder or an increased risk of the neurological disorder when the sample comprises a higher ratio of SM (d43:1)/SM (d34:1) compared to the control sample. In another aspect, the one or more biomarker metabolites comprises one or more ceramides (Cer). In another aspect, the subject is determined as having the neurological disorder or an increased risk of the neurological disorder when the sample comprises higher concentration levels of one or more Cer or a higher Cer/SM ratio compared to the control sample. In another aspect, the method further comprises treating the subject with a therapeutically effective amount of a compound capable of modulating the concentration levels or ratios of the one or more biomarker metabolites related to ceramide or sphingomyelin metabolism to reduce the negative effects of the neurological disorder. In one aspect, the compound modulates the concentration levels or ratios of one or more sphingolipids in the subject. In another aspect, the compound increases the concentration levels of sphingosine-1-phosphate (S1P), modulates signaling by S1P, or a combination thereof. In another aspect, the compound modulates the ratio of SM (d43:1)/SM (d34:1). In another aspect, the compound decreases the concentration levels of one or more Cer and/or the ratio of Cer/SM. In another aspect, the compound comprises a sphingosine-1-phosphate receptor (S1PR) modulator including one or more of fingolimod, SEW2871, siponimod, ozanimod, ceralifimod, GSK2018682, ponesimod, KRP203, cenerimod, amiselimod (MT-1303), etrasimod, laquinimod, derivatives thereof, or combinations thereof. In another aspect, the compound comprises fingolimod or a derivative thereof. In another aspect, treating the subject comprises orally administering the compound. In another aspect, the neurological disorder is a neuropsychiatric or neurodegenerative symptom associated with neurological diseases or cognitive impairment, including depression, anxiety, dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's disease, young-onset dementia, Korsakoffs syndrome, Huntington's disease, HIV-associated neurocognitive disorders, multiple sclerosis, or other cognitive impairment, neuropsychiatric, or neurodegenerative disorders.

Another embodiment described herein is a method for detecting and treating a neurological disorder in a subject, the method comprising: analyzing a sample from a subject and a control sample from a control subject or population of subjects with normal cognition; measuring the concentration levels or ratios of one or more biomarker metabolites related to ceramide or sphingomyelin metabolism in the sample from the subject and the control sample; determining the subject as having a neurological disorder or an increased risk of a neurological disorder when the concentration levels or ratios of the one or more biomarker metabolites in the sample from the subject are different from (greater than or less than) the concentration levels or ratios of the one or more biomarker metabolites in the control sample; and treating the subject with a therapeutically effective amount of a compound capable of modulating the concentration levels or ratios of the one or more biomarker metabolites related to ceramide or sphingomyelin metabolism to reduce the negative effects of the neurological disorder. In another aspect, the one or more biomarker metabolites related to ceramide or sphingomyelin metabolism comprises one or more sphingolipids including: sphingomyelins (SM): SM (OH) C14:1 (SM C15:0); SM C16:0; SM (OH) C16:1 (SM C17:0); SM C16:1; SM C18:0; SM C18:1; SM C20:2; SM (OH) C22:1 (SM C23:0); SM (OH) C22:2 (SM C23:1); SM C24:0; SM (OH) C24:1 (SM C25:0); SM C24:1; SM C26:0; SM C26:1; SM (d43:1); SM (d34:1); SM (d32:0); SM (d38:3); SM (d33:0); SM (d38:2); SM (d33:1); SM (d32:1); SM (d44:1); SM (d42:1); SM (d41:1); SM (d35:1); SM (d36:1); SM (d44:2); SM (d34:2); SM (d36:2); or combinations thereof; ceramides (Cer): Cer (d18:1,14:0); Cer (d18:1,16:1); Cer (d18:0,16:1); Cer (d16:1,16:0); Cer (d18:2,16:0); Cer (d18:1,16:0); Cer (d18:0,16:0); Cer (d16:0,18:1); Cer (d16:1,18:0); Cer (d18:1,18:0); Cer (d18:1, 22:1); Cer (d18:0, 22:1); Cer (d18:2, 22:0); Cer (d18:1, 22:0); Cer (d18:1, 23:1); Cer (d18:0, 23:1); Cer (d18:2, 23:0); Cer (d18:1, 23:0); Cer (d18:1, 24:2); Cer (d18:0, 24:2); Cer (d16:1, 24:1); Cer (d18:2, 24:1); Cer (d18:1, 24:1); Cer (d18:0, 24:1); Cer (d16:1, 24:0); Cer (d18:2, 24:0); Cer (d18:1, 24:0); Cer (d18:0, 24:0); Cer (d20:1, 24:0); Cer (d20:0, 24:0); Cer (d18:1, 26:1); Cer (d18:0, 26:1); Cer (d18:2, 26:0); Cer (d18:1, 26:0); Cer (d18:0, 26:0); or combinations thereof; or sphingosine-1-phosphate (S1P). In another aspect, the one or more biomarker metabolites comprises S1P. In another aspect, the one or more biomarker metabolites related to ceramide or sphingomyelin metabolism comprises one or more sphingomyelins (SM): SM (OH) C14:1 (SM C15:0); SM C16:0; SM (OH) C16:1 (SM C17:0); SM C16:1; SM C18:0; SM C18:1; SM C20:2; SM (OH) C22:1 (SM C23:0); SM (OH) C22:2 (SM C23:1); SM C24:0; SM (OH) C24:1 (SM C25:0); SM C24:1; SM C26:0; SM C26:1; SM (d43:1); SM (d34:1); SM (d32:0); SM (d38:3); SM (d33:0); SM (d38:2); SM (d33:1); SM (d32:1); SM (d44:1); SM (d42:1); SM (d41:1); SM (d35:1); SM (d36:1); SM (d44:2); SM (d34:2); SM (d36:2); or combinations thereof; and wherein one or more ratios of the SM is calculated. In another aspect, the one or more biomarker metabolites related to ceramide or sphingomyelin metabolism comprises SM (d43:1) and SM (d34:1), and wherein a ratio of SM (d43:1)/SM (d34:1) is calculated. In another aspect, the subject is determined as having the neurological disorder or an increased risk of the neurological disorder when the sample comprises a higher ratio of SM (d43:1)/SM (d34:1) compared to the control sample. In another aspect, the one or more biomarker metabolites comprises one or more ceramides (Cer). In another aspect, the compound comprises fingolimod, SEW2871, siponimod, ozanimod, ceralifimod, GSK2018682, ponesimod, KRP203, cenerimod, amiselimod (MT-1303), etrasimod, laquinimod, derivatives thereof, or combinations thereof. In another aspect, the compound comprises fingolimod or a derivative thereof. In another aspect, the neurological disorder is a neuropsychiatric or neurodegenerative symptom associated with neurological diseases or cognitive impairment, including depression, anxiety, dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's disease, young-onset dementia, Korsakoff's syndrome, Huntington's disease, HIV-associated neurocognitive disorders, multiple sclerosis, or other cognitive impairment, neuropsychiatric, or neurodegenerative disorders.

Another embodiment described herein is a method for detecting a neurological disorder in a subject, the method comprising: analyzing a sample from a subject and a control sample from a control subject or population of subjects with normal cognition; measuring the concentration levels of one or more enzymes related to ceramide or sphingomyelin metabolism in the sample from the subject and the control sample; and determining the subject as having a neurological disorder or an increased risk of a neurological disorder when the concentration levels of the one or more enzymes in the sample from the subject are different from (greater than or less than) the concentration levels of the one or more enzymes in the control sample. In another aspect, the one or more enzymes related to ceramide or sphingomyelin metabolism comprises one or more of serine palmitoyltransferase (SPTLC1; SPTLC2; SPTLC3), sphingomyelin synthase (SGMS1; SGMS2), sphingomyelin phosphodiesterase (SMPD1; SMPD2), ceramide kinase (CERK), phosphatidate phosphatase (PLPP2), ceramidase (ASAH1), ceramide synthase (CERS2; CERS3; CERS4), sphingosine kinase (SPHK1; SPHK2), sphingosine-1-phosphate phosphatase (SGPP1), sphingosine-1-phosphate lyase (SGPL1), or combinations thereof. In another aspect, the enzyme comprises sphingomyelin synthase (SGMS1). In another aspect, the one or more enzymes related to ceramide or sphingomyelin metabolism comprises one or more enzymes involved in ceramide or sphingomyelin synthesis including serine palmitoyltransferase (SPTLC3) and ceramide synthase (CERS2; CERS4); one or more enzymes involved in sphingosine-1-phosphate synthesis and degradation including sphingosine kinase (SPHK2), sphingosine-1-phosphate phosphatase (SGPP1), and sphingosine-1-phosphate lyase (SGPL1); or combinations thereof. In another aspect, the method further comprises performing neuroimaging analysis on the subject and the control subject or population of subjects with normal cognition, and correlating the results of the neuroimaging analysis with the measured concentration levels of the one or more enzymes related to ceramide or sphingomyelin metabolism in the sample from the subject and the control sample, to link the results of the neuroimaging analysis to the subject's metabolic profile. In another aspect, the neuroimaging analysis assesses brain atrophy, brain glucose metabolism, or a combination thereof. In another aspect, the neuroimaging analysis comprises structural magnetic resonance imaging (MRI), molecular [¹⁸F] fluorodeoxyglucose (FDG) positron emission tomography (PET), or a combination thereof. In another aspect, the method further comprises treating the subject with a therapeutically effective amount of a compound capable of modulating the concentration levels of the one or more enzymes related to ceramide or sphingomyelin metabolism to reduce the negative effects of the neurological disorder. In another aspect, the compound comprises a sphingosine-1-phosphate receptor (S1PR) modulator including one or more of fingolimod, SEW2871, siponimod, ozanimod, ceralifimod, GSK2018682, ponesimod, KRP203, cenerimod, amiselimod (MT-1303), etrasimod, laquinimod, derivatives thereof, or combinations thereof. In another aspect, the compound comprises fingolimod or a derivative thereof. In another aspect, the neurological disorder is a neuropsychiatric or neurodegenerative symptom associated with neurological diseases or cognitive impairment, including depression, anxiety, dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's disease, young-onset dementia, Korsakoff's syndrome, Huntington's disease, HIV-associated neurocognitive disorders, multiple sclerosis, or other cognitive impairment, neuropsychiatric, or neurodegenerative disorders.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows an overview of the sphingolipid pathway manually curated from the Recon3D model. The metabolites participating in reactions are represented in boxes. The arrows for reactions A-K are colored based on the direction in the pathway. Some reactions are not reversible (single arrows). FIG. 1B shows a table listing the catalyzing enzymes in the sphingolipid pathway in humans. The enzymes are denoted with the same color code as the reaction arrow in FIG. 1A.

FIG. 2A-C show box plots of reaction fluxes for (FIG. 2A) serine palmitoyl transferase (SPT), (FIG. 2B) sphingomyelin synthase (SMS), and (FIG. 2C) ceramide kinase (CERK) reactions. The orange, green, blue, and purple bars correspond to Alzheimer's disease (AD), mild cognitive impairment (MCI), normal (NC), and other dementia, respectively.

FIG. 3A-F show the association of genetic variants in SPTLC3 and SGMS1 with structural (MRI) and molecular (FDG-PET) neuroimaging phenotypes. FIG. 3A shows gene-based association analysis of SPTLC3 with cognitive performance (Rey auditory verbal learning test total score). FIG. 3B and FIG. 3E show surface-based whole brain analysis of cortical thickness (brain atrophy measured from MRI scans) for SPTLC3 and SGMS1, respectively. FIG. 3C and FIG. 3F show voxel-based whole-brain analysis of brain glucose metabolism measured from FDG-PET scans for SPTLC3 and SGMS1, respectively. FIG. 3D shows gene-based association analysis of SGMS1 with global brain glucose metabolism.

FIG. 4 shows a hybrid network of genetic associations revealed by gene-based association studies and significant partial correlations of detected sphingomyelins. The six identified genes can be grouped into two categories: global sphingomyelin (SM) synthesis; and synthesis and degradation of sphingosine-1-phosphate (S1P). The selected SM ratio is colored in orange, other SM species are in green (light green: non-targeted metabolomics; dark green: targeted metabolomics), and genes are in dark yellow.

FIG. 5A-F show a comparison of WT and APP/PS1 mice hippocampal dependent behavior and synaptic transmission assessment. FIG. 5A shows exploration time spent on the novel object in a NOR test session. Data are expressed as a discrimination index ±SEM. FIG. 5B shows Barnes Maze task performance during training days. Acquisition learning trials were performed, and the time it took to locate and enter into the escape box (i.e., latency) was recorded in seconds. The average performance of four trials per day was expressed as mean±SEM. A shorter latency indicates faster spatial learning. FIG. 5C shows data from a probe trial that was performed on day 5 of the Barnes Maze protocol, during which the escape box was removed. The percentage of time spent inside the target quadrant (the previous escape box location) is plotted ±SEM. A larger percentage of time indicates better spatial memory. FIG. 5D shows an LTP timeline. Plotted are normalized evoked excitatory post-synaptic potentials (EPSPs) slopes (y-axis) vs. recording time (x-axis). The first 20 min of evoked responses were normalized and used as the baseline responses of LTP. FIG. 5E shows representative analog traces of evoked EPSPs before (light purple and grey) and after (blue and green) high frequency stimulation (HFS). FIG. 5F shows that the magnitude of LTP was determined according to the responses between 45 and 60 min after the HFS. Data represent mean fEPSP Slope ±SEM (n=6 mice in each group).

FIG. 6A-F show the effect of fingolimod treatment on WT and APP/PS1 mice. FIG. 6A shows data of WT and APP/PS1 mice treated with fingolimod that were tested in the NOR task. Data are expressed as a discrimination index ±SEM. Fingolimod treatment significantly enhanced the discrimination index of the APP/PS1 mice. FIG. 6B shows Barnes Maze task performance during training days where the average performance of four trials per day is expressed as mean±SEM. FIG. 6C shows the percentage of time spent inside the target quadrant ±SEM at the Barnes Maze task. Fingolimod treatment mitigated the spatial learning deficits of the APP/PS1 mice at 9 m.o. FIG. 6D shows LTP of the CA3 to CA1 synapse timeline. FIG. 6E shows representative analog traces of evoked EPSPs before (light blue and light red) and after (blue and red) HFS. FIG. 6F shows that fingolimod treatment in the APP/PS1 mice group significantly augmented the normalized slope of fEPSP after HFS.

FIG. 7 shows a study overview. Information was obtained from post-mortem brain transcriptomics analysis; metabolic networks of brain regions; genetic variants associated with AD biomarkers including neuroimaging endophenotypes (MRI and FDG PET); plasma metabolomics and lipidomics analysis; and genetic screening using SM (d43:1)/SM (d34:1) ratio. The balance of ceramide and sphingomyelin levels in AD could be maintained by modulating S1PR activity. The study hypothesis was tested in APP/PS1 mice treated with fingolimod.

### DETAILED DESCRIPTION

Unless otherwise defined, all technical and scientific terms used herein have the same meaning as commonly understood by one of ordinary skill in the art. For example, any nomenclatures used in connection with, and techniques of biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

As used herein, “treatment,” “therapy,” or “therapy regimen” also refer to the clinical intervention made in response to a disease, disorder, or physiological condition (e.g., a depressive disorder) manifested by a patient or to which a patient may be susceptible. The aim of treatment includes the alleviation or prevention of symptoms, slowing or stopping the progression or worsening of a disease, disorder, or condition and/or the remission of the disease, disorder, or condition (e.g., a depressive disorder). As used herein, the terms “prevent,” “preventing,” “prevention,” “prophylactic treatment” and the like refer to reducing the probability of developing a disease, disorder or condition in a subject, who does not have, but is at risk of or susceptible to developing a disease, disorder or condition. The term “effective amount” or “therapeutically effective amount” refers to an amount sufficient to effect beneficial or desirable biological and/or clinical results.

As used herein, the term “administering” an agent, such as a therapeutic entity to treat a depressive disorder to an animal or cell, is intended to refer to dispensing, delivering, or applying the substance to the intended target. In terms of the therapeutic agent, the term “administering” is intended to refer to contacting or dispensing, delivering or applying the therapeutic agent to a subject by any suitable route for delivery of the therapeutic agent to the desired location in the animal, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route.

As used herein, the term “biomarker” refers to a naturally occurring biological molecule present in a subject at varying concentrations useful in predicting the risk or incidence of a disease or a condition, such as a depressive disorder. For example, the biomarker can be a protein present in higher or lower amounts in a subject at risk for a depressive disorder. The biomarker can include nucleic acids, ribonucleic acids, metabolite, protein, fatty acid, lipid, polypeptide or the like that is used as an indicator or marker for a depressive disorder in the subject. Biomarkers may reflect a variety of disease characteristics, including the level of exposure to an environmental or genetic trigger, an element of the disease process itself, and intermediate stage between exposure and disease onset, or an independent factor associated with the disease state, but not causative of pathogenesis. Biomarkers may be used to determine the status of a subject or the effectiveness of a treatment. Biomarker combinations with the most diagnostic utility have both high sensitivity and specificity. In practice, biomarkers and/or specific combinations of biomarkers having both high sensitivity and specificity are not obvious. Evaluation, assessment, and combination of specific biomarkers for diagnosis provide an improved approach to disease treatment.

The term “biological sample” as used herein includes, but is not limited to, a sample containing tissues, cells, including peripheral cells in human blood, and/or biological fluids isolated from a subject. Examples of biological samples include, but are not limited to, tissues, cells, biopsies, muscle, interstitial fluid, sweat, saliva, urine, tears, synovial fluid, bone marrow, cerebrospinal fluid, nasal secretions, sputum, mucus, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In one embodiment, the biological sample comprises a blood sample. A biological sample may be obtained directly from a subject (e.g., by blood or tissue sampling) or from a third party (e.g., received from an intermediary, such as a healthcare provider or lab technician).

As used herein, the terms “neurological diseases” or “neurological disorders” are used interchangeably and refer to a host of undesirable conditions affecting neurons in the brain of a subject and can include neurodegenerative and neuropsychiatric diseases and disorders. Representative examples of such conditions include, without limitation, Alzheimer's disease, Parkinson's disease, Huntington's disease, Pick's disease, Kufs disease, Lewy body disease, neurofibrillary tangles, Rosenthal fibers, Mallory's hyaline, senile dementia, myasthenia gravis, Gilles de la Tourette's syndrome, multiple sclerosis (MS), amyotrophic lateral sclerosis (ALS), progressive supranuclear palsy (PSP), epilepsy, Creutzfeldt-Jakob disease, deafness-dystonia syndrome, Leigh syndrome, Leber hereditary optic neuropathy (LHON), parkinsonism, dystonia, motor neuron disease, neuropathy-ataxia and retinitis pigmentosa (NARP), maternal inherited Leigh syndrome (MILS), Friedreich ataxia, hereditary spastic paraplegia, Mohr-Tranebjaerg syndrome, Wilson disease, sporadic Alzheimer's disease, sporadic amyotrophic lateral sclerosis, sporadic Parkinson's disease, autonomic function disorders, hypertension, sleep disorders, neuropsychiatric disorders, depression, schizophrenia, schizoaffective disorder, Korsakoff's psychosis, mania, anxiety disorders, phobic disorder, learning or memory disorders, amnesia or age-related memory loss, attention deficit disorder, dysthymic disorder, major depressive disorder, obsessive-compulsive disorder, psychoactive substance use disorders, panic disorder, bipolar affective disorder, severe bipolar affective (mood) disorder (BP-1), migraines, hyperactivity and movement disorders.

As used herein, the term “movement disorder” includes neurological diseases or disorders that involve the motor and movement systems, resulting in a range of abnormalities that affect the speed, quality, and ease of movement. Movement disorders are often caused by or related to abnormalities in brain structure and/or function. Movement disorders include, but are not limited to (i) tremors: including, but not limited to, the tremor associated with Parkinson's Disease, physiologic tremor, benign familial tremor, cerebellar tremor, rubral tremor, toxic tremor, metabolic tremor, and senile tremor; (ii) chorea, including, but not limited to, chorea associated with Huntington's Disease, Wilson's Disease, ataxia telangiectasia, infection, drug ingestion, or metabolic, vascular or endocrine etiology (e.g., chorea gravidarum or thyrotoxicosis); (iii) ballism (defined herein as abruptly beginning, repetitive, wide, flinging movements affecting predominantly the proximal limb and girdle muscles); (iv) athetosis (defined herein as relatively slow, twisting, writhing, snake-like movements and postures involving the trunk, neck, face and extremities); (v) dystonia (defined herein as a movement disorder consisting of twisting, turning tonic skeletal muscle contractions, most, but not all of which are initiated distally); (vi) paroxysmal choreoathetosis and tonic spasm; (vii) tics (defined herein as sudden, behaviorally related, irregular, stereotyped, repetitive movements of variable complexity); (viii) tardive dyskinesia; (ix) akathisia, (x) muscle rigidity, defined herein as resistance of a muscle to stretch; (xi) postural instability; (xii) bradykinesia; (xiii) difficulty in initiating movements; (xiv) muscle cramps; (xv) dyskinesias and (xvi) myoclonus. In some embodiments, the neurological disorder comprises dystonia.

As used herein, “depression” refers to a neuropsychiatric mood disorder that causes a persistent feeling of sadness, anxiety, and loss of interest. As used herein “depression” includes subclinical characteristics associated with depression such as anxiety, sadness, loss of interest in activities, loss of appetite, anhedonia, insomnia, changes in sleep, difficulty falling asleep, waking during the night, restless sleep, waking too early, sleeping too much, low energy level, lack of concentration, diminished or altered daily behavior, low self-esteem, suicidal thoughts, or anxiety coupled with depression. Included in this definition is Major Depression Disorder (MDD), core depression (CD+), anxious depression (ANX+), neurovegetative symptoms of melancholia (NVSM+), treatment resistant depression, or subclinical characteristics associated with depression.

Treatments for depressive disorders may include any of those currently available treatments and can be readily determined by one skilled in the art. Such treatments may include antidepressants, including but not limited to, ketamine, tranylcypromine, phenelzine, selegiline, isocarboxazid, amitriptyline, clomipramine, desipramine, doxepin, imipramine, nortryptyline, amoxapine, protriptyline, trimipramine, bupropion, nefazodone, venlafaxine, mirtazapine, duloxetine, fluoxetine, fluvoxamine, paroxetine, sertraline, citalopram, or escitalopram. In one aspect, the antidepressant is one or more selective serotonin reuptake inhibitors (SSRI). In another aspect, the antidepressant comprises an SSRI selected from escitalopram, citalopram, fluoxetine, sertraline, paroxetine, fluvoxamine, vilazodone, vortioxetine, or duloxetine. In another aspect, the antidepressant comprises an SSRI comprising escitalopram or citalopram.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The methods and compositions disclosed herein can be used on a sample either in vitro (for example, on isolated cells or tissues) or in vivo in a subject (i.e., living organism, such as a patient).

Lipids, including sphingomyelin (SM), have been shown to be disrupted in AD. However, the impacts of lipid alterations on AD pathogenesis are not fully understood. Brain lipids constitute ˜50% of the brain's dry weight with myelin, a proteolipid, composed of 70-80% lipids. Previous evidence implicates various sphingolipids in neuronal signaling and toxicity. SM is one of the abundant sphingolipid classes present in the central nervous system (CNS) and primarily resides in two locations within the brain: (1) lipid rafts, found in neurons, astrocytes, and microglia where they are involved in several aspects of signal transduction and homeostasis of the brain; and (2) the membranous myelin sheath that insulates many nerve cell axons. As part of the myelin sheath and lipid rafts, SMs are involved in signal transduction and the regulation of inflammatory processes and response to oxidative stress. Previous studies have indicated a complex pattern of deregulation in sphingolipid metabolism, including ceramides, in the early stages of AD, and have also reported changes at the gene expression level of the myelin network in AD.

The hydrolysis of SM produces ceramide (Cer). Ceramides are the simplest of sphingolipids, are neurotoxic, and induce apoptosis. Ceramides mediate the relationship between Aβ and neurodegeneration. Increasing Aβ levels elevates SM phosphodiesterase (SMase) activity, leading to an increase in Cer. It is suggested that the increase in ceramides boosts BACE-1 activity, which cleaves APP in two sequential steps to produce soluble Ap. Furthermore, sphingosine-1-phosphate (S1P) is an important neuroprotective signaling molecule and product of the SM pathway, as S1P blocks SMase activity and inhibits amyloid precursor protein (APP) secretion. By understanding the changes in SM/Cer ratios and their underlying mechanism, it will allow for a better understanding how perturbations in the SM pathway contributes to neurodegeneration.

As part of normal homeostasis, microglia constantly surveil the brain parenchyma. During development, and throughout normal lifespan, microglia remove neuronal synapses, eliminate dying neurons, and clean up myelin debris. Sphingolipid-rich neuronal and myelin membranes captured through these processes undergo lysosomal degradation within microglia. This degradative process is facilitated by a lipid-sensing receptor, TREM2, that is activated by various lipid, including sphingolipids, sphingomyelin, and sulfatide. TREM2-deficient microglia phagocytose myelin debris but fail to clear myelin cholesterol, resulting in cholesteryl ester (CE) accumulation. A CE increase is also observed in APOE-deficient glial cells, reflecting impaired brain cholesterol transport. Recent studies have begun to elucidate the important role of microglia in AD, with evidence for differences in microglial subpopulations, related to myelin clearance and activation.

Genome-wide and transcriptome-wide association studies have identified various genes and genetic variants in lipid metabolism that are associated with AD. However, the molecular mechanisms of SM and Cer disruption remain to be determined. Evaluation of peripheral lipidomic profiles is useful in providing perspective on metabolic dysregulation in preclinical and clinical AD states.

Described herein are studies focused on the sphingolipid pathway using multi-omic analyses to identify central and peripheral metabolic changes in AD patients and correlate them to imaging features and cognitive performance in amyloidogenic mouse models. These studies used human in vivo data and post-mortem brain data to finely characterize the SM pathway for molecular links to AD pathogenesis. Metabolic readouts were identified that can be utilized to link observed molecular changes back to potential intervention targets, which were experimentally validated in animal models resulting in repurposed drug for AD. Gene expression profiling from three large cohorts was used to identify differentially expressed genes in the SM pathway. Constraint-based modeling was then used to narrow down the search space to identify potential changes in metabolic fluxes between cognitively normal and AD individuals. Next, genetic variants were identified within the SM pathway and their association was assessed with changes in neuroimaging using a large multicenter biomarker study. Then, changes in plasma lipidomic species were identified within the SM pathway with specific SNPs using data from the ADNI and AIBL cohort.

The multi-omic approach was based on (a) 2114 human post-mortem brain transcriptomics to identify differentially expressed genes; (b) in silico metabolic flux analysis on 1708 context-specific metabolic networks to identify differential reaction fluxes; (c) multimodal neuroimaging analysis on 1576 participants to associate genetic variants in SM pathway with AD pathogenesis; (d) plasma metabolomic and lipidomic analysis to identify associations of lipid species with dysregulation in AD; and (e) metabolite genome-wide association studies (mGWAS) to define receptors within the pathway as potential drug targets.

Using these complementary approaches, S1P was identified as regulating the balance in the SM pathway. Findings from these complementary approaches suggest that depletion of S1P compensates for AD cellular pathology, likely by upregulating the SM pathway, suggesting that modulation of S1P signaling may have protective effects in AD. This hypothesis was tested in APP/PS1 mice and demonstrated that prolonged exposure to fingolimod, an S1P receptor (S1PR) modulator that causes internalization of S1PR and is approved for treatment of multiple sclerosis, alleviated the cognitive impairment in the mice. Therefore, these studies indicate that S1P is a metabolite involved in maintaining the balance in the SM pathway and represents a target for identifying drugs to modulate S1P metabolism and levels for treatment of AD.

S1P Receptor (S1PR) Modulators Oral S1PR modulators are FDA-approved for the treatment of relapsing, remitting multiple sclerosis (RR-MS), and are in clinical trials for other inflammatory diseases (Table 1). S1PR modulators are typically synthetic structural analogs of sphingosine and are phosphorylated by SPHKs in vivo to activate S1PR. While some agonists activate multiple S1P receptor types (e.g., fingolimod), others are selective for the S1P1 receptor (S1P1R). Fingolimod (Gilenya® Novartis), the first oral drug approved for MS, is thought to function in MS by preventing lymphocyte egress from lymphoid tissues. Treatment with fingolimod reduces the infiltration of lymphocytes into the CNS, preventing inflammation and tissue damage. Fingolimod also has direct effects on S1P signaling. S1P binding results in internalization and recycling of the S1P1R. Phosphorylated fingolimod causes prolonged internalization and degradation of the receptor, thus acting as a functional antagonist. Also, fingolimod affects S1PRs on neurons and glial cells, and has been implicated in decreased production of cytokines and enhanced expression of brain-derived neurotrophic factor. Fingolimod treatment results in enhanced synaptic function and anti-apoptotic activity, reducing the production and the neurotoxicity of Aβ peptides and promoting the survival of microglia and neurons.

TABLE 1
S1P Receptor (S1PR) Modulators

| | | Indication | Approved |
|---|---|---|---|
| S1PR drugs | Target | for diseases | for use |
| Fingolimod- | S1P1, S1P3, | RR-MS, Stroke | US FDA/EU |
| FTY720 | S1P4, S1P5 | | EMA |
| Siponimod- | S1P1, S1P5 | SP-MS, RR-MS | US FDA/EU |
| BAF312 | | | EMA |
| Ozanimod- | S1P1 | RR-MS, UC, | US FDA/EU |
| RPC1063 | | Crohn's | EMA |
| Ceralifimod- | S1P1, S1P5 | RR-MS | Discontinued |
| ONO-4641 | | | |
| GSK2018682 | S1P1 | RR-MS | Discontinued |
| Ponesimod- | S1P1 | RR-MS | NDA |
| ACT-128800 | | | submitted |
| KRP203 | S1P1 | U. colitis, SLE | Discontinued |
| Cenerimod- | S1P1 | SLE | In clinical |
| ACT-33441 | | | trials |
| Amiselimod- | S1P1, S1P4, | RR-MS, Crohn's, | In clinical |
| MT1303 | S1P5 | Psoriasis | trials |
| Etrasimod- | S1P1, S1P4, | Ulcerative colitis | In clinical |
| APD334 | S1P5 | | trials |
| Laquinimod | S1P1 | Multiple sclerosis | Russia |
| | | (MS) | |

Selective S1PR modulators improve cognitive function in clinical trials of RR-MS. Tests show a benefit in favor of Siponimod compared with placebo for Symbol Digit Modalities Test (SDMT) oral score at study months 12 and 24. While there were no statistically significant differences between groups for PASAT score or Brief Visuospatial Memory Test-Revised, for the low contrast visual acuity test, there was a small benefit in favor of Siponimod. Ozanimod treatment was associated with improved cognitive processing speed as assessed by the Symbol Digit Modalities Test and slower loss of thalamic volume as compared to IFNβ1a treatment. S1PR modulators have had neurologically related outcomes measured in five multiple sclerosis clinical trials (Table 2).

TABLE 2
Clinical Trial Measures of Cognition using S1PR Modulators

| | | |
|---|---|---|
| S1PR Drugs/ | Patients | |
| Clinical Trial | Enrolled | Cognitive-related Outcome |

Fingolimod

| | | |
|---|---|---|
| FREEDOMS II | 249 Treated, | Brain volume |
| | 257 Placebo | |

Siponimod

| | | |
|---|---|---|
| Expand Trial | 1105 Treated, | Neurological Exams |
| | 546 Placebo | |

Ozanimod

| | | |
|---|---|---|
| ENLIGHTEN | 250 Total | Cognitive processing speed, |
| | | Brain volume |
| SUNBEAM | 1346 Total | Cognitive processing speed |

Ponesimod

| | | |
|---|---|---|
| OPTIMUM | 1133 Total | Brain volume |

Some embodiments of the present disclosure are based, in part, on research using an integrated multi-omics approach, using independent datasets to finely characterize the SM pathway for molecular links to AD pathogenesis and to identify metabolic readouts that can be utilized to link observed molecular changes back to potential intervention targets. Efforts started with analyzing the gene expression profiles from post-mortem brain samples to identify differentially expressed genes in the SM pathway. Using a constraint-based modeling approach, potential changes in metabolic flux between cognitively normal and AD individuals were identified. Next, variants were identified in the genes of the SM pathway with Amyloid, Tau, Neurodegeneration, Cognition (A-T-N-C) measures of AD by examining the genetic associations of CSF biomarker levels, brain atrophy (magnetic resonance imaging), brain glucose metabolism ([¹⁸F] FDG PET), cognition, and clinical diagnosis. Then, plasma metabolomics and lipidomic profiles were examined and changes in the ratio of SM species to be associated with sphingolipid dysregulation in AD were identified. The ratio of SM species was used as an intermediate for genetic screening and to identify markers in the SM pathway. This analysis highlighted a potential role for S1P metabolism and signaling in AD. Further embodiments relate to the deregulation of S1P metabolism in amyloidogenic APP/PS1 mice and the demonstration that the repurposed drug, fingolimod, was able to improve cognition in mice, presumably through impacting the SM pathway.

Accordingly, aspects and embodiments of the present disclosure provide methods for identifying, diagnosing, or screening a subject for a neurological disorder, such as AD, comprising, consisting of, or consisting essentially of obtaining a biological sample from a subject, determining the levels of S1P in the sample as compared to a control in which decreased levels of S1P are indicative of the neurological disorder, and administering to the subject a therapeutic amount of a therapy suitable for the treatment of the neurological disorder.

Another aspect of the present disclosure provides a method of treating a subject suffering from a neurological disorder comprising, consisting of, or consisting essentially of administering to the subject a therapeutically effective amount of a compound capable of modulating the levels of S1P in the subject. In some embodiments, the compound comprises fingolimod.

One embodiment described herein is a method for the classification and treatment of a neurological disorder in a subject based on the subject's metabolic profile and genetic screening, the method comprising one or more of the following: identifying and stratifying subjects afflicted with a neurological disorder to subgroups based on their metabolic profiles, biomarker metabolites and ratios of biomarker metabolites that define unique metabolic conditions related to aberrations in ceramide and sphingomyelin anabolism, catabolism, or homeostasis and common identity among subgroups of subjects; evaluating the trajectory of disease within each stratified subgroup of subjects and their response to a therapeutic treatment; identifying defects in ceramide or sphingomyelin transport and/or biosynthesis of biomarker metabolites within a metabolic pathway or across metabolic pathways using ratios of biomarker metabolites to inform about changes in enzyme activities or transporters; and identifying genetic bases of ceramide and sphingomyelin metabolic profile characteristics or defects (SNPs/genetic variants in key enzymes and transporters) using metabolite genome-wide association study (mGWAS) analysis. In one aspect, the method further comprises one or more of the following: using combined metabotype and genotype data and genome-scale metabolic models to better stratify subjects with the neurological disorder and to inform about mechanisms and treatment selection; suggesting a therapeutic approach to correct metabolic defects in metabolic profile in stratified subgroups of subjects; and comparing and contrasting metabolic defects noted in inborn errors of metabolism that have the neurological disorder and using knowledge gained in treatment of inborn errors of metabolism to inform treatment for neurological disorder. In another aspect, the method further comprises administering to the subjects an effective amount of a therapy to prevent and/or treat the neurological disorder affected by one or more metabolic defects. In another aspect, the neurological disorder is a neuropsychiatric or neurodegenerative symptom associated with neurological diseases or cognitive impairment, including depression, anxiety, dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's disease, young-onset dementia, Korsakoff's syndrome, Huntington's disease, HIV-associated neurocognitive disorders, multiple sclerosis, or other cognitive impairment, neuropsychiatric, or neurodegenerative disorders.

Another embodiment described herein is a method for stratifying and treating a subject having a neurological disorder, or at risk of developing a neurological disorder, based on the subject's metabolic profile, the method comprising: analyzing a sample from a subject to determine concentration levels or ratios of one or more biomarker metabolites related to ceramide and sphingomyelin anabolism, catabolism, or homeostasis in the sample from the subject; determining if the subject has a metabolic defect related to disrupted ceramide and sphingomyelin anabolism, catabolism, or homeostasis, or if the subject's gut microbiome has a defect related to disrupted ceramide and sphingomyelin anabolism, catabolism, or homeostasis, or combinations thereof based on the measured concentration levels and calculated ratios of the one or more ceramide and sphingomyelin anabolism, catabolism, or homeostasis biomarker metabolites in the sample as compared to a control sample; stratifying the subject into a subgroup of subjects, wherein an individual subgroup of subjects is defined by a unique and specific ceramide and sphingomyelin anabolism, catabolism, or homeostasis profile based on the measured concentration levels and calculated ratios of the one or more biomarker metabolites in the sample as compared to a control sample and the biomarker metabolite defect determined for the subject. In one aspect, the method further comprises treating the neurological disorder by administering to the subgroup of subjects an effective amount of a therapy sufficient to attenuate, reduce, or eliminate the symptoms of neurological disorder, wherein the therapy is determined by the unique and specific metabolic profile of the subgroup of subjects. In another aspect, the one or more biomarker metabolites comprises one or more of: sphingomyelins (SM): SM (OH) C14:1 (SM C15:0); SM C16:0; SM (OH) C16:1 (SM C17:0); SM C16:1; SM C18:0; SM C18:1; SM C20:2; SM (OH) C22:1 (SM C23:0); SM (OH) C22:2 (SM C23:1); SM C24:0; SM (OH) C24:1 (SM C25:0); SM C24:1; SM C26:0; SM C26:1; SM (d43:1); SM (d34:1); SM (d32:0); SM (d38:3); SM (d33:0); SM (d38:2); SM (d33:1); SM (d32:1); SM (d44:1); SM (d42:1); SM (d41:1); SM (d35:1); SM (d36:1); SM (d44:2); SM (d34:2); SM (d36:2); or combinations thereof; ceramides (Cer): Cer (d18:1,14:0); Cer (d18:1,16:1); Cer (d18:0,16:1); Cer (d16:1,16:0); Cer (d18:2,16:0); Cer (d18:1,16:0); Cer (d18:0,16:0); Cer (d16:0,18:1); Cer (d16:1,18:0); Cer (d18:1,18:0); Cer (d18:1, 22:1); Cer (d18:0, 22:1); Cer (d18:2, 22:0); Cer (d18:1, 22:0); Cer (d18:1, 23:1); Cer (d18:0, 23:1); Cer (d18:2, 23:0); Cer (d18:1, 23:0); Cer (d18:1, 24:2); Cer (d18:0, 24:2); Cer (d16:1, 24:1); Cer (d18:2, 24:1); Cer (d18:1, 24:1); Cer (d18:0, 24:1); Cer (d16:1, 24:0); Cer (d18:2, 24:0); Cer (d18:1, 24:0); Cer (d18:0, 24:0); Cer (d20:1, 24:0); Cer (d20:0, 24:0); Cer (d18:1, 26:1); Cer (d18:0, 26:1); Cer (d18:2, 26:0); Cer (d18:1, 26:0); Cer (d18:0, 26:0); or combinations thereof; or sphingosine-1-phosphate (S1P). In another aspect, the one or more biomarker metabolites comprises S1P. In another aspect, the one or more biomarker metabolites related to ceramide or sphingomyelin metabolism comprises one or more sphingomyelins (SM): SM (OH) C14:1 (SM C15:0); SM C16:0; SM (OH) C16:1 (SM C17:0); SM C16:1; SM C18:0; SM C18:1; SM C20:2; SM (OH) C22:1 (SM C23:0); SM (OH) C22:2 (SM C23:1); SM C24:0; SM (OH) C24:1 (SM C25:0); SM C24:1; SM C26:0; SM C26:1; SM (d43:1); SM (d34:1); SM (d32:0); SM (d38:3); SM (d33:0); SM (d38:2); SM (d33:1); SM (d32:1); SM (d44:1); SM (d42:1); SM (d41:1); SM (d35:1); SM (d36:1); SM (d44:2); SM (d34:2); SM (d36:2); or combinations thereof; and wherein one or more ratios of the SM is calculated. In another aspect, the one or more biomarker metabolites related to ceramide or sphingomyelin metabolism comprises SM (d43:1) and SM (d34:1), and wherein a ratio of SM (d43:1)/SM (d34:1) is calculated. In another aspect, the subject is determined as having the neurological disorder or an increased risk of the neurological disorder when the sample comprises a higher ratio of SM (d43:1)/SM (d34:1) compared to the control sample. In another aspect, the one or more biomarker metabolites comprises one or more ceramides (Cer). In another aspect, the method further comprises: administering to the subject a therapeutically effective amount of one or more ceramides, sphingomyelins, and/or any pharmaceutically acceptable derivatives, esters, salts, solvates, hydrates, analogs, or prodrugs thereof; and/or administering to the subject a therapeutically effective amount of one or more therapeutic agents capable of modulating (increasing or decreasing) the concentration levels or ratios of one or more primary or sphingomyelins, activating the endogenous production of one or more ceramides or sphingomyelins, and/or decreasing the breakdown of one or more ceramides or sphingomyelins; and/or administering to the subject a therapeutically effective amount of a sphingosine-1-phosphate receptor (S1PR) modulator including one or more of fingolimod, SEW2871, siponimod, ozanimod, ceralifimod, GSK2018682, ponesimod, KRP203, cenerimod, amiselimod (MT-1303), etrasimod, laquinimod, derivatives thereof, or combinations thereof.

Another embodiment described herein is a method for detecting a neurological disorder in a subject, the method comprising: analyzing a sample from a subject and a control sample from a control subject or population of subjects with normal cognition; measuring the concentration levels or ratios of one or more biomarker metabolites related to ceramide or sphingomyelin metabolism in the sample from the subject and the control sample; and determining the subject as having a neurological disorder or an increased risk of a neurological disorder when the concentration levels or ratios of the one or more biomarker metabolites in the sample from the subject are different from (greater than or less than) the concentration levels or ratios of the one or more biomarker metabolites in the control sample. In one aspect, the one or more biomarker metabolites related to ceramide or sphingomyelin metabolism comprises one or more sphingolipids including: sphingomyelins (SM): SM (OH) C14:1 (SM C15:0); SM C16:0; SM (OH) C16:1 (SM C17:0); SM C16:1; SM C18:0; SM C18:1; SM C20:2; SM (OH) C22:1 (SM C23:0); SM (OH) C22:2 (SM C23:1); SM C24:0; SM (OH) C24:1 (SM C25:0); SM C24:1; SM C26:0; SM C26:1; SM (d43:1); SM (d34:1); SM (d32:0); SM (d38:3); SM (d33:0); SM (d38:2); SM (d33:1); SM (d32:1); SM (d44:1); SM (d42:1); SM (d41:1); SM (d35:1); SM (d36:1); SM (d44:2); SM (d34:2); SM (d36:2); or combinations thereof; ceramides (Cer): Cer (d18:1,14:0); Cer (d18:1,16:1); Cer (d18:0,16:1); Cer (d16:1,16:0); Cer (d18:2,16:0); Cer (d18:1,16:0); Cer (d18:0,16:0); Cer (d16:0,18:1); Cer (d16:1,18:0); Cer (d18:1,18:0); Cer (d18:1, 22:1); Cer (d18:0, 22:1); Cer (d18:2, 22:0); Cer (d18:1, 22:0); Cer (d18:1, 23:1); Cer (d18:0, 23:1); Cer (d18:2, 23:0); Cer (d18:1, 23:0); Cer (d18:1, 24:2); Cer (d18:0, 24:2); Cer (d16:1, 24:1); Cer (d18:2, 24:1); Cer (d18:1, 24:1); Cer (d18:0, 24:1); Cer (d16:1, 24:0); Cer (d18:2, 24:0); Cer (d18:1, 24:0); Cer (d18:0, 24:0); Cer (d20:1, 24:0); Cer (d20:0, 24:0); Cer (d18:1, 26:1); Cer (d18:0, 26:1); Cer (d18:2, 26:0); Cer (d18:1, 26:0); Cer (d18:0, 26:0); or combinations thereof; or sphingosine-1-phosphate (S1P). In another aspect, the one or more biomarker metabolites comprises S1P. In another aspect, the subject is determined as having the neurological disorder or an increased risk of the neurological disorder when the sample comprises lower concentration levels of S1P compared to the control sample. In another aspect, the one or more biomarker metabolites related to ceramide or sphingomyelin metabolism comprises one or more sphingomyelins (SM): SM (OH) C14:1 (SM C15:0); SM C16:0; SM (OH) C16:1 (SM C17:0); SM C16:1; SM C18:0; SM C18:1; SM C20:2; SM (OH) C22:1 (SM C23:0); SM (OH) C22:2 (SM C23:1); SM C24:0; SM (OH) C24:1 (SM C25:0); SM C24:1; SM C26:0; SM C26:1; SM (d43:1); SM (d34:1); SM (d32:0); SM (d38:3); SM (d33:0); SM (d38:2); SM (d33:1); SM (d32:1); SM (d44:1); SM (d42:1); SM (d41:1); SM (d35:1); SM (d36:1); SM (d44:2); SM (d34:2); SM (d36:2); or combinations thereof; and wherein one or more ratios of the SM is calculated. In another aspect, the one or more biomarker metabolites related to ceramide or sphingomyelin metabolism comprises SM (d43:1) and SM (d34:1), and wherein a ratio of SM (d43:1)/SM (d34:1) is calculated. In another aspect, the subject is determined as having the neurological disorder or an increased risk of the neurological disorder when the sample comprises a higher ratio of SM (d43:1)/SM (d34:1) compared to the control sample. In another aspect, the one or more biomarker metabolites comprises one or more ceramides (Cer). In another aspect, the subject is determined as having the neurological disorder or an increased risk of the neurological disorder when the sample comprises higher concentration levels of one or more Cer or a higher Cer/SM ratio compared to the control sample. In another aspect, the method further comprises treating the subject with a therapeutically effective amount of a compound capable of modulating the concentration levels or ratios of the one or more biomarker metabolites related to ceramide or sphingomyelin metabolism to reduce the negative effects of the neurological disorder. In one aspect, the compound modulates the concentration levels or ratios of one or more sphingolipids in the subject. In another aspect, the compound increases the concentration levels of sphingosine-1-phosphate (S1P), modulates signaling by S1P, or a combination thereof. In another aspect, the compound modulates the ratio of SM (d43:1)/SM (d34:1). In another aspect, the compound decreases the concentration levels of one or more Cer and/or the ratio of Cer/SM. In another aspect, the compound comprises a sphingosine-1-phosphate receptor (S1PR) modulator including one or more of fingolimod, SEW2871, siponimod, ozanimod, ceralifimod, GSK2018682, ponesimod, KRP203, cenerimod, amiselimod (MT-1303), etrasimod, laquinimod, derivatives thereof, or combinations thereof. In another aspect, the compound comprises fingolimod or a derivative thereof. In another aspect, treating the subject comprises orally administering the compound. In another aspect, the neurological disorder is a neuropsychiatric or neurodegenerative symptom associated with neurological diseases or cognitive impairment, including depression, anxiety, dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's disease, young-onset dementia, Korsakoff's syndrome, Huntington's disease, HIV-associated neurocognitive disorders, multiple sclerosis, or other cognitive impairment, neuropsychiatric, or neurodegenerative disorders.

Another embodiment described herein is a method for detecting and treating a neurological disorder in a subject, the method comprising: analyzing a sample from a subject and a control sample from a control subject or population of subjects with normal cognition; measuring the concentration levels or ratios of one or more biomarker metabolites related to ceramide or sphingomyelin metabolism in the sample from the subject and the control sample; determining the subject as having a neurological disorder or an increased risk of a neurological disorder when the concentration levels or ratios of the one or more biomarker metabolites in the sample from the subject are different from (greater than or less than) the concentration levels or ratios of the one or more biomarker metabolites in the control sample; and treating the subject with a therapeutically effective amount of a compound capable of modulating the concentration levels or ratios of the one or more biomarker metabolites related to ceramide or sphingomyelin metabolism to reduce the negative effects of the neurological disorder. In another aspect, the one or more biomarker metabolites related to ceramide or sphingomyelin metabolism comprises one or more sphingolipids including: sphingomyelins (SM): SM (OH) C14:1 (SM C15:0); SM C16:0; SM (OH) C16:1 (SM C17:0); SM C16:1; SM C18:0; SM C18:1; SM C20:2; SM (OH) C22:1 (SM C23:0); SM (OH) C22:2 (SM C23:1); SM C24:0; SM (OH) C24:1 (SM C25:0); SM C24:1; SM C26:0; SM C26:1; SM (d43:1); SM (d34:1); SM (d32:0); SM (d38:3); SM (d33:0); SM (d38:2); SM (d33:1); SM (d32:1); SM (d44:1); SM (d42:1); SM (d41:1); SM (d35:1); SM (d36:1); SM (d44:2); SM (d34:2); SM (d36:2); or combinations thereof; ceramides (Cer): Cer (d18:1,14:0); Cer (d18:1,16:1); Cer (d18:0,16:1); Cer (d16:1,16:0); Cer (d18:2,16:0); Cer (d18:1,16:0); Cer (d18:0,16:0); Cer (d16:0,18:1); Cer (d16:1,18:0); Cer (d18:1,18:0); Cer (d18:1, 22:1); Cer (d18:0, 22:1); Cer (d18:2, 22:0); Cer (d18:1, 22:0); Cer (d18:1, 23:1); Cer (d18:0, 23:1); Cer (d18:2, 23:0); Cer (d18:1, 23:0); Cer (d18:1, 24:2); Cer (d18:0, 24:2); Cer (d16:1, 24:1); Cer (d18:2, 24:1); Cer (d18:1, 24:1); Cer (d18:0, 24:1); Cer (d16:1, 24:0); Cer (d18:2, 24:0); Cer (d18:1, 24:0); Cer (d18:0, 24:0); Cer (d20:1, 24:0); Cer (d20:0, 24:0); Cer (d18:1, 26:1); Cer (d18:0, 26:1); Cer (d18:2, 26:0); Cer (d18:1, 26:0); Cer (d18:0, 26:0); or combinations thereof; or sphingosine-1-phosphate (S1P). In another aspect, the one or more biomarker metabolites comprises S1P. In another aspect, the one or more biomarker metabolites related to ceramide or sphingomyelin metabolism comprises one or more sphingomyelins (SM): SM (OH) C14:1 (SM C15:0); SM C16:0; SM (OH) C16:1 (SM C17:0); SM C16:1; SM C18:0; SM C18:1; SM C20:2; SM (OH) C22:1 (SM C23:0); SM (OH) C22:2 (SM C23:1); SM C24:0; SM (OH) C24:1 (SM C25:0); SM C24:1; SM C26:0; SM C26:1; SM (d43:1); SM (d34:1); SM (d32:0); SM (d38:3); SM (d33:0); SM (d38:2); SM (d33:1); SM (d32:1); SM (d44:1); SM (d42:1); SM (d41:1); SM (d35:1); SM (d36:1); SM (d44:2); SM (d34:2); SM (d36:2); or combinations thereof; and wherein one or more ratios of the SM is calculated. In another aspect, the one or more biomarker metabolites related to ceramide or sphingomyelin metabolism comprises SM (d43:1) and SM (d34:1), and wherein a ratio of SM (d43:1)/SM (d34:1) is calculated. In another aspect, the subject is determined as having the neurological disorder or an increased risk of the neurological disorder when the sample comprises a higher ratio of SM (d43:1)/SM (d34:1) compared to the control sample. In another aspect, the one or more biomarker metabolites comprises one or more ceramides (Cer). In another aspect, the compound comprises fingolimod, SEW2871, siponimod, ozanimod, ceralifimod, GSK2018682, ponesimod, KRP203, cenerimod, amiselimod (MT-1303), etrasimod, laquinimod, derivatives thereof, or combinations thereof. In another aspect, the compound comprises fingolimod or a derivative thereof. In another aspect, the neurological disorder is a neuropsychiatric or neurodegenerative symptom associated with neurological diseases or cognitive impairment, including depression, anxiety, dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's disease, young-onset dementia, Korsakoffs syndrome, Huntington's disease, HIV-associated neurocognitive disorders, multiple sclerosis, or other cognitive impairment, neuropsychiatric, or neurodegenerative disorders.

Another embodiment described herein is a method for detecting a neurological disorder in a subject, the method comprising: analyzing a sample from a subject and a control sample from a control subject or population of subjects with normal cognition; measuring the concentration levels of one or more enzymes related to ceramide or sphingomyelin metabolism in the sample from the subject and the control sample; and determining the subject as having a neurological disorder or an increased risk of a neurological disorder when the concentration levels of the one or more enzymes in the sample from the subject are different from (greater than or less than) the concentration levels of the one or more enzymes in the control sample. In another aspect, the one or more enzymes related to ceramide or sphingomyelin metabolism comprises one or more of serine palmitoyltransferase (SPTLC1; SPTLC2; SPTLC3), sphingomyelin synthase (SGMS1; SGMS2), sphingomyelin phosphodiesterase (SMPD1; SMPD2), ceramide kinase (CERK), phosphatidate phosphatase (PLPP2), ceramidase (ASAH1), ceramide synthase (CERS2; CERS3; CERS4), sphingosine kinase (SPHK1; SPHK2), sphingosine-1-phosphate phosphatase (SGPP1), sphingosine-1-phosphate lyase (SGPL1), or combinations thereof. In another aspect, the enzyme comprises sphingomyelin synthase (SGMS1). In another aspect, the one or more enzymes related to ceramide or sphingomyelin metabolism comprises one or more enzymes involved in ceramide or sphingomyelin synthesis including serine palmitoyltransferase (SPTLC3) and ceramide synthase (CERS2; CERS4); one or more enzymes involved in sphingosine-1-phosphate synthesis and degradation including sphingosine kinase (SPHK2), sphingosine-1-phosphate phosphatase (SGPP1), and sphingosine-1-phosphate lyase (SGPL1); or combinations thereof. In another aspect, the method further comprises performing neuroimaging analysis on the subject and the control subject or population of subjects with normal cognition, and correlating the results of the neuroimaging analysis with the measured concentration levels of the one or more enzymes related to ceramide or sphingomyelin metabolism in the sample from the subject and the control sample, to link the results of the neuroimaging analysis to the subject's metabolic profile. In another aspect, the neuroimaging analysis assesses brain atrophy, brain glucose metabolism, or a combination thereof. In another aspect, the neuroimaging analysis comprises structural magnetic resonance imaging (MRI), molecular [¹⁸F] fluorodeoxyglucose (FDG) positron emission tomography (PET), or a combination thereof. In another aspect, the method further comprises treating the subject with a therapeutically effective amount of a compound capable of modulating the concentration levels of the one or more enzymes related to ceramide or sphingomyelin metabolism to reduce the negative effects of the neurological disorder. In another aspect, the compound comprises a sphingosine-1-phosphate receptor (S1PR) modulator including one or more of fingolimod, SEW2871, siponimod, ozanimod, ceralifimod, GSK2018682, ponesimod, KRP203, cenerimod, amiselimod (MT-1303), etrasimod, laquinimod, derivatives thereof, or combinations thereof. In another aspect, the compound comprises fingolimod or a derivative thereof. In another aspect, the neurological disorder is a neuropsychiatric or neurodegenerative symptom associated with neurological diseases or cognitive impairment, including depression, anxiety, dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's disease, young-onset dementia, Korsakoff's syndrome, Huntington's disease, HIV-associated neurocognitive disorders, multiple sclerosis, or other cognitive impairment, neuropsychiatric, or neurodegenerative disorders.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A method for the classification and treatment of a neurological disorder in a subject based on the subject's metabolic profile and genetic screening, the method comprising one or more of the following:
   identifying and stratifying subjects afflicted with a neurological disorder to subgroups based on their metabolic profiles, biomarker metabolites and ratios of biomarker metabolites that define unique metabolic conditions related to aberrations in ceramide and sphingomyelin anabolism, catabolism, or homeostasis and common identity among subgroups of subjects;
   evaluating the trajectory of disease within each stratified subgroup of subjects and their response to a therapeutic treatment;
   identifying defects in ceramide or sphingomyelin transport and/or biosynthesis of biomarker metabolites within a metabolic pathway or across metabolic pathways using ratios of biomarker metabolites to inform about changes in enzyme activities or transporters; and
   identifying genetic bases of ceramide and sphingomyelin metabolic profile characteristics or defects (SNPs/genetic variants in key enzymes and transporters) using metabolite genome-wide association study (mGWAS) analysis.
Clause 2. The method of clause 1, further comprising one or more of the following:
   using combined metabotype and genotype data and genome-scale metabolic models to better stratify subjects with the neurological disorder and to inform about mechanisms and treatment selection;
   suggesting a therapeutic approach to correct metabolic defects in metabolic profile in stratified subgroups of subjects; and
   comparing and contrasting metabolic defects noted in inborn errors of metabolism that have the neurological disorder and using knowledge gained in treatment of inborn errors of metabolism to inform treatment for neurological disorder.
Clause 3. The method of clause 1 or 2, further comprising administering to the subjects an effective amount of a therapy to prevent and/or treat the neurological disorder affected by one or more metabolic defects.
Clause 4. The method of any one of clauses 1-3, wherein the neurological disorder is a neuropsychiatric or neurodegenerative symptom associated with neurological diseases or cognitive impairment, including depression, anxiety, dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's disease, young-onset dementia, Korsakoff's syndrome, Huntington's disease, HIV-associated neurocognitive disorders, multiple sclerosis, or other cognitive impairment, neuropsychiatric, or neurodegenerative disorders.
Clause 5. A method for stratifying and treating a subject having a neurological disorder, or at risk of developing a neurological disorder, based on the subject's metabolic profile, the method comprising:
   analyzing a sample from a subject to determine concentration levels or ratios of one or more biomarker metabolites related to ceramide and sphingomyelin anabolism, catabolism, or homeostasis in the sample from the subject;
   determining if the subject has a metabolic defect related to disrupted ceramide and sphingomyelin anabolism, catabolism, or homeostasis, or if the subject's gut microbiome has a defect related to disrupted ceramide and sphingomyelin anabolism, catabolism, or homeostasis, or combinations thereof based on the measured concentration levels and calculated ratios of the one or more ceramide and sphingomyelin anabolism, catabolism, or homeostasis biomarker metabolites in the sample as compared to a control sample;
   stratifying the subject into a subgroup of subjects, wherein an individual subgroup of subjects is defined by a unique and specific ceramide and sphingomyelin anabolism, catabolism, or homeostasis profile based on the measured concentration levels and calculated ratios of the one or more biomarker metabolites in the sample as compared to a control sample and the biomarker metabolite defect determined for the subject.
Clause 6. The method of clause 5, further comprising treating the neurological disorder by administering to the subgroup of subjects an effective amount of a therapy sufficient to attenuate, reduce, or eliminate the symptoms of neurological disorder, wherein the therapy is determined by the unique and specific metabolic profile of the subgroup of subjects.
Clause 7. The method of clause 5 or 6, wherein the one or more biomarker metabolites comprises one or more of:
   sphingomyelins (SM): SM (OH) C14:1 (SM C15:0); SM C16:0; SM (OH) C16:1 (SM C17:0); SM C16:1; SM C18:0; SM C18:1; SM C20:2; SM (OH) C22:1 (SM C23:0); SM (OH) C22:2 (SM C23:1); SM C24:0; SM (OH) C24:1 (SM C25:0); SM C24:1; SM C26:0; SM C26:1; SM (d43:1); SM (d34:1); SM (d32:0); SM (d38:3); SM (d33:0); SM (d38:2); SM (d33:1); SM (d32:1); SM (d44:1); SM (d42:1); SM (d41:1); SM (d35:1); SM (d36:1); SM (d44:2); SM (d34:2); SM (d36:2); or combinations thereof;
   ceramides (Cer): Cer (d18:1,14:0); Cer (d18:1,16:1); Cer (d18:0,16:1); Cer (d16:1,16:0); Cer (d18:2,16:0); Cer (d18:1,16:0); Cer (d18:0,16:0); Cer (d16:0,18:1); Cer (d16:1,18:0); Cer (d18:1,18:0); Cer (d18:1, 22:1); Cer (d18:0, 22:1); Cer (d18:2, 22:0); Cer (d18:1, 22:0); Cer (d18:1, 23:1); Cer (d18:0, 23:1); Cer (d18:2, 23:0); Cer (d18:1, 23:0); Cer (d18:1, 24:2); Cer (d18:0, 24:2); Cer (d16:1, 24:1); Cer (d18:2, 24:1); Cer (d18:1, 24:1); Cer (d18:0, 24:1); Cer (d16:1, 24:0); Cer (d18:2, 24:0); Cer (d18:1, 24:0); Cer (d18:0, 24:0); Cer (d20:1, 24:0); Cer (d20:0, 24:0); Cer (d18:1, 26:1); Cer (d18:0, 26:1); Cer (d18:2, 26:0); Cer (d18:1, 26:0); Cer (d18:0, 26:0); or combinations thereof; or
   sphingosine-1-phosphate (S1P).
Clause 8. The method of any one of clauses 5-7, wherein the one or more biomarker metabolites comprises S1P.
Clause 9. The method of any one of clauses 5-8, wherein the one or more biomarker metabolites related to ceramide or sphingomyelin metabolism comprises one or more sphingomyelins (SM): SM (OH) C14:1 (SM C15:0); SM C16:0; SM (OH) C16:1 (SM C17:0); SM C16:1; SM C18:0; SM C18:1; SM C20:2; SM (OH) C22:1 (SM C23:0); SM (OH) C22:2 (SM C23:1); SM C24:0; SM (OH) C24:1 (SM C25:0); SM C24:1; SM C26:0; SM C26:1; SM (d43:1); SM (d34:1); SM (d32:0); SM (d38:3); SM (d33:0); SM (d38:2); SM (d33:1); SM (d32:1); SM (d44:1); SM (d42:1); SM (d41:1); SM (d35:1); SM (d36:1); SM (d44:2); SM (d34:2); SM (d36:2); or combinations thereof; and wherein one or more ratios of the SM is calculated.
Clause 10. The method of any one of clauses 5-9, wherein the one or more biomarker metabolites related to ceramide or sphingomyelin metabolism comprises SM (d43:1) and SM (d34:1), and wherein a ratio of SM (d43:1)/SM (d34:1) is calculated.
Clause 11. The method of any one of clauses 5-10, wherein the subject is determined as having the neurological disorder or an increased risk of the neurological disorder when the sample comprises a higher ratio of SM (d43:1)/SM (d34:1) compared to the control sample.
Clause 12. The method of any one of clauses 5-11, wherein the one or more biomarker metabolites comprises one or more ceramides (Cer).
Clause 13. The method of any one of clauses 5-12, further comprising:
   administering to the subject a therapeutically effective amount of one or more ceramides, sphingomyelins, and/or any pharmaceutically acceptable derivatives, esters, salts, solvates, hydrates, analogs, or prodrugs thereof; and/or
   administering to the subject a therapeutically effective amount of one or more therapeutic agents capable of modulating (increasing or decreasing) the concentration levels or ratios of one or more primary or sphingomyelins, activating the endogenous production of one or more ceramides or sphingomyelins, and/or decreasing the breakdown of one or more ceramides or sphingomyelins; and/or
   administering to the subject a therapeutically effective amount of a sphingosine-1-phosphate receptor (S1PR) modulator including one or more of fingolimod, SEW2871, siponimod, ozanimod, ceralifimod, GSK2018682, ponesimod, KRP203, cenerimod, amiselimod (MT-1303), etrasimod, laquinimod, derivatives thereof, or combinations thereof.
Clause 14. A method for detecting a neurological disorder in a subject, the method comprising:
   analyzing a sample from a subject and a control sample from a control subject or population of subjects with normal cognition;
   measuring the concentration levels or ratios of one or more biomarker metabolites related to ceramide or sphingomyelin metabolism in the sample from the subject and the control sample; and
   determining the subject as having a neurological disorder or an increased risk of a neurological disorder when the concentration levels or ratios of the one or more biomarker metabolites in the sample from the subject are different from (greater than or less than) the concentration levels or ratios of the one or more biomarker metabolites in the control sample.
Clause 15. The method of clause 14, wherein the one or more biomarker metabolites related to ceramide or sphingomyelin metabolism comprises one or more sphingolipids including:
   sphingomyelins (SM): SM (OH) C14:1 (SM C15:0); SM C16:0; SM (OH) C16:1 (SM C17:0); SM C16:1; SM C18:0; SM C18:1; SM C20:2; SM (OH) C22:1 (SM C23:0); SM (OH) C22:2 (SM C23:1); SM C24:0; SM (OH) C24:1 (SM C25:0); SM C24:1; SM C26:0; SM C26:1; SM (d43:1); SM (d34:1); SM (d32:0); SM (d38:3); SM (d33:0); SM (d38:2); SM (d33:1); SM (d32:1); SM (d44:1); SM (d42:1); SM (d41:1); SM (d35:1); SM (d36:1); SM (d44:2); SM (d34:2); SM (d36:2); or combinations thereof;
   ceramides (Cer): Cer (d18:1,14:0); Cer (d18:1,16:1); Cer (d18:0,16:1); Cer (d16:1,16:0); Cer (d18:2,16:0); Cer (d18:1,16:0); Cer (d18:0,16:0); Cer (d16:0,18:1); Cer (d16:1,18:0); Cer (d18:1,18:0); Cer (d18:1, 22:1); Cer (d18:0, 22:1); Cer (d18:2, 22:0); Cer (d18:1, 22:0); Cer (d18:1, 23:1); Cer (d18:0, 23:1); Cer (d18:2, 23:0); Cer (d18:1, 23:0); Cer (d18:1, 24:2); Cer (d18:0, 24:2); Cer (d16:1, 24:1); Cer (d18:2, 24:1); Cer (d18:1, 24:1); Cer (d18:0, 24:1); Cer (d16:1, 24:0); Cer (d18:2, 24:0); Cer (d18:1, 24:0); Cer (d18:0, 24:0); Cer (d20:1, 24:0); Cer (d20:0, 24:0); Cer (d18:1, 26:1); Cer (d18:0, 26:1); Cer (d18:2, 26:0); Cer (d18:1, 26:0); Cer (d18:0, 26:0); or combinations thereof; or
   sphingosine-1-phosphate (S1P).
Clause 16. The method of clause 14 or 15, wherein the one or more biomarker metabolites comprises S1P.
Clause 17. The method of any one of clauses 14-16, wherein the subject is determined as having the neurological disorder or an increased risk of the neurological disorder when the sample comprises lower concentration levels of S1P compared to the control sample.
Clause 18. The method of any one of clauses 14-17, wherein the one or more biomarker metabolites related to ceramide or sphingomyelin metabolism comprises one or more sphingomyelins (SM): SM (OH) C14:1 (SM C15:0); SM C16:0; SM (OH) C16:1 (SM C17:0); SM C16:1; SM C18:0; SM C18:1; SM C20:2; SM (OH) C22:1 (SM C23:0); SM (OH) C22:2 (SM C23:1); SM C24:0; SM (OH) C24:1 (SM C25:0); SM C24:1; SM C26:0; SM C26:1; SM (d43:1); SM (d34:1); SM (d32:0); SM (d38:3); SM (d33:0); SM (d38:2); SM (d33:1); SM (d32:1); SM (d44:1); SM (d42:1); SM (d41:1); SM (d35:1); SM (d36:1); SM (d44:2); SM (d34:2); SM (d36:2); or combinations thereof; and wherein one or more ratios of the SM is calculated.
Clause 19. The method of any one of clauses 14-18, wherein the one or more biomarker metabolites related to ceramide or sphingomyelin metabolism comprises SM (d43:1) and SM (d34:1), and wherein a ratio of SM (d43:1)/SM (d34:1) is calculated.
Clause 20. The method of any one of clauses 14-19, wherein the subject is determined as having the neurological disorder or an increased risk of the neurological disorder when the sample comprises a higher ratio of SM (d43:1)/SM (d34:1) compared to the control sample.
Clause 21. The method of any one of clauses 14-20, wherein the one or more biomarker metabolites comprises one or more ceramides (Cer).
Clause 22. The method of any one of clauses 14-21, wherein the subject is determined as having the neurological disorder or an increased risk of the neurological disorder when the sample comprises higher concentration levels of one or more Cer or a higher Cer/SM ratio compared to the control sample.
Clause 23. The method of any one of clauses 14-22, further comprising treating the subject with a therapeutically effective amount of a compound capable of modulating the concentration levels or ratios of the one or more biomarker metabolites related to ceramide or sphingomyelin metabolism to reduce the negative effects of the neurological disorder.
Clause 24. The method of any one of clauses 14-23 wherein the compound modulates the concentration levels or ratios of one or more sphingolipids in the subject.
Clause 25. The method of any one of clauses 14-24, wherein the compound increases the concentration levels of sphingosine-1-phosphate (S1P), modulates signaling by S1P, or a combination thereof.
Clause 26. The method of any one of clauses 14-25, wherein the compound modulates the ratio of SM (d43:1)/SM (d34:1).
Clause 27. The method of any one of clauses 14-26, wherein the compound decreases the concentration levels of one or more Cer and/or the ratio of Cer/SM.
Clause 28. The method of any one of clauses 14-27, wherein the compound comprises a sphingosine-1-phosphate receptor (S1PR) modulator including one or more of fingolimod, SEW2871, siponimod, ozanimod, ceralifimod, GSK2018682, ponesimod, KRP203, cenerimod, amiselimod (MT-1303), etrasimod, laquinimod, derivatives thereof, or combinations thereof.
Clause 29. The method of any one of clauses 14-28, wherein the compound comprises fingolimod or a derivative thereof.
Clause 30. The method of any one of clauses 14-29, wherein treating the subject comprises orally administering the compound.
Clause 31. The method of any one of clauses 14-30, wherein the neurological disorder is a neuropsychiatric or neurodegenerative symptom associated with neurological diseases or cognitive impairment, including depression, anxiety, dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's disease, young-onset dementia, Korsakoff's syndrome, Huntington's disease, HIV-associated neurocognitive disorders, multiple sclerosis, or other cognitive impairment, neuropsychiatric, or neurodegenerative disorders.
Clause 32. A method for detecting and treating a neurological disorder in a subject, the method comprising:
   analyzing a sample from a subject and a control sample from a control subject or population of subjects with normal cognition;
   measuring the concentration levels or ratios of one or more biomarker metabolites related to ceramide or sphingomyelin metabolism in the sample from the subject and the control sample;
   determining the subject as having a neurological disorder or an increased risk of a neurological disorder when the concentration levels or ratios of the one or more biomarker metabolites in the sample from the subject are different from (greater than or less than) the concentration levels or ratios of the one or more biomarker metabolites in the control sample; and
   treating the subject with a therapeutically effective amount of a compound capable of modulating the concentration levels or ratios of the one or more biomarker metabolites related to ceramide or sphingomyelin metabolism to reduce the negative effects of the neurological disorder.
Clause 33. The method of clause 32, wherein the one or more biomarker metabolites related to ceramide or sphingomyelin metabolism comprises one or more sphingolipids including:
   sphingomyelins (SM): SM (OH) C14:1 (SM C15:0); SM C16:0; SM (OH) C16:1 (SM C17:0); SM C16:1; SM C18:0; SM C18:1; SM C20:2; SM (OH) C22:1 (SM C23:0); SM (OH) C22:2 (SM C23:1); SM C24:0; SM (OH) C24:1 (SM C25:0); SM C24:1; SM C26:0; SM C26:1; SM (d43:1); SM (d34:1); SM (d32:0); SM (d38:3); SM (d33:0); SM (d38:2); SM (d33:1); SM (d32:1); SM (d44:1); SM (d42:1); SM (d41:1); SM (d35:1); SM (d36:1); SM (d44:2); SM (d34:2); SM (d36:2); or combinations thereof;
   ceramides (Cer): Cer (d18:1,14:0); Cer (d18:1,16:1); Cer (d18:0,16:1); Cer (d16:1,16:0); Cer (d18:2,16:0); Cer (d18:1,16:0); Cer (d18:0,16:0); Cer (d16:0,18:1); Cer (d16:1,18:0); Cer (d18:1,18:0); Cer (d18:1, 22:1); Cer (d18:0, 22:1); Cer (d18:2, 22:0); Cer (d18:1, 22:0); Cer (d18:1, 23:1); Cer (d18:0, 23:1); Cer (d18:2, 23:0); Cer (d18:1, 23:0); Cer (d18:1, 24:2); Cer (d18:0, 24:2); Cer (d16:1, 24:1); Cer (d18:2, 24:1); Cer (d18:1, 24:1); Cer (d18:0, 24:1); Cer (d16:1, 24:0); Cer (d18:2, 24:0); Cer (d18:1, 24:0); Cer (d18:0, 24:0); Cer (d20:1, 24:0); Cer (d20:0, 24:0); Cer (d18:1, 26:1); Cer (d18:0, 26:1); Cer (d18:2, 26:0); Cer (d18:1, 26:0); Cer (d18:0, 26:0); or combinations thereof; or
   sphingosine-1-phosphate (S1P).
Clause 34. The method of clause 32 or 33, wherein the one or more biomarker metabolites comprises S1P.
Clause 35. The method of any one of clauses 32-35, wherein the one or more biomarker metabolites related to ceramide or sphingomyelin metabolism comprises one or more sphingomyelins (SM): SM (OH) C14:1 (SM C15:0); SM C16:0; SM (OH) C16:1 (SM C17:0); SM C16:1; SM C18:0; SM C18:1; SM C20:2; SM (OH) C22:1 (SM C23:0); SM (OH) C22:2 (SM C23:1); SM C24:0; SM (OH) C24:1 (SM C25:0); SM C24:1; SM C26:0; SM C26:1; SM (d43:1); SM (d34:1); SM (d32:0); SM (d38:3); SM (d33:0); SM (d38:2); SM (d33:1); SM (d32:1); SM (d44:1); SM (d42:1); SM (d41:1); SM (d35:1); SM (d36:1); SM (d44:2); SM (d34:2); SM (d36:2); or combinations thereof; and wherein one or more ratios of the SM is calculated.
Clause 36. The method of any one of clauses 32-35, wherein the one or more biomarker metabolites related to ceramide or sphingomyelin metabolism comprises SM (d43:1) and SM (d34:1), and wherein a ratio of SM (d43:1)/SM (d34:1) is calculated.
Clause 37. The method of any one of clauses 32-36, wherein the subject is determined as having the neurological disorder or an increased risk of the neurological disorder when the sample comprises a higher ratio of SM (d43:1)/SM (d34:1) compared to the control sample.
Clause 38. The method of any one of clauses 32-37, wherein the one or more biomarker metabolites comprises one or more ceramides (Cer).
Clause 39. The method of any one of clauses 32-38, wherein the compound comprises fingolimod, SEW2871, siponimod, ozanimod, ceralifimod, GSK2018682, ponesimod, KRP203, cenerimod, amiselimod (MT-1303), etrasimod, laquinimod, derivatives thereof, or combinations thereof.
Clause 40. The method any one of clauses 32-39, wherein the compound comprises fingolimod or a derivative thereof.
Clause 41. The method of any one of clauses 32-40, wherein the neurological disorder is a neuropsychiatric or neurodegenerative symptom associated with neurological diseases or cognitive impairment, including depression, anxiety, dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's disease, young-onset dementia, Korsakoff's syndrome, Huntington's disease, HIV-associated neurocognitive disorders, multiple sclerosis, or other cognitive impairment, neuropsychiatric, or neurodegenerative disorders.
Clause 42. A method for detecting a neurological disorder in a subject, the method comprising:
   analyzing a sample from a subject and a control sample from a control subject or population of subjects with normal cognition;
   measuring the concentration levels of one or more enzymes related to ceramide or sphingomyelin metabolism in the sample from the subject and the control sample; and
   determining the subject as having a neurological disorder or an increased risk of a neurological disorder when the concentration levels of the one or more enzymes in the sample from the subject are different from (greater than or less than) the concentration levels of the one or more enzymes in the control sample.
Clause 43. The method of clause 42, wherein the one or more enzymes related to ceramide or sphingomyelin metabolism comprises one or more of serine palmitoyltransferase (SPTLC1; SPTLC2; SPTLC3), sphingomyelin synthase (SGMS1; SGMS2), sphingomyelin phosphodiesterase (SMPD1; SMPD2), ceramide kinase (CERK), phosphatidate phosphatase (PLPP2), ceramidase (ASAH1), ceramide synthase (CERS2; CERS3; CERS4), sphingosine kinase (SPHK1; SPHK2), sphingosine-1-phosphate phosphatase (SGPP1), sphingosine-1-phosphate lyase (SGPL1), or combinations thereof.
Clause 44. The method of clause 42 or 43, wherein the enzyme comprises sphingomyelin synthase (SGMS1).
Clause 45. The method of any one of clauses 42-44, wherein the one or more enzymes related to ceramide or sphingomyelin metabolism comprises one or more enzymes involved in ceramide or sphingomyelin synthesis including serine palmitoyltransferase (SPTLC3) and ceramide synthase (CERS2; CERS4); one or more enzymes involved in sphingosine-1-phosphate synthesis and degradation including sphingosine kinase (SPHK2), sphingosine-1-phosphate phosphatase (SGPP1), and sphingosine-1-phosphate lyase (SGPL1); or combinations thereof.
Clause 46. The method of any one of clauses 42-45, further comprising performing neuroimaging analysis on the subject and the control subject or population of subjects with normal cognition, and correlating the results of the neuroimaging analysis with the measured concentration levels of the one or more enzymes related to ceramide or sphingomyelin metabolism in the sample from the subject and the control sample, to link the results of the neuroimaging analysis to the subject's metabolic profile.
Clause 47. The method of any one of clauses 42-46, wherein the neuroimaging analysis assesses brain atrophy, brain glucose metabolism, or a combination thereof.
Clause 48. The method of any one of clauses 42-47, wherein the neuroimaging analysis comprises structural magnetic resonance imaging (MRI), molecular [¹⁸F]fluorodeoxyglucose (FDG) positron emission tomography (PET), or a combination thereof.
Clause 49. The method of any one of clauses 42-48, further comprising treating the subject with a therapeutically effective amount of a compound capable of modulating the concentration levels of the one or more enzymes related to ceramide or sphingomyelin metabolism to reduce the negative effects of the neurological disorder.
Clause 50. The method of any one of clauses 42-49, wherein the compound comprises a sphingosine-1-phosphate receptor (S1PR) modulator including one or more of fingolimod, SEW2871, siponimod, ozanimod, ceralifimod, GSK2018682, ponesimod, KRP203, cenerimod, amiselimod (MT-1303), etrasimod, laquinimod, derivatives thereof, or combinations thereof.
Clause 51. The method of any one of clauses 42-50, wherein the compound comprises fingolimod or a derivative thereof.
Clause 52. The method of any one of clauses 42-51, wherein the neurological disorder is a neuropsychiatric or neurodegenerative symptom associated with neurological diseases or cognitive impairment, including depression, anxiety, dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's disease, young-onset dementia, Korsakoff's syndrome, Huntington's disease, HIV-associated neurocognitive disorders, multiple sclerosis, or other cognitive impairment, neuropsychiatric, or neurodegenerative disorders.

### EXAMPLES

### Example 1

### Identification of Differential Gene Expression in Brain Tissue RNA-Seq Data

The reprocessed AMP-AD RNA-seq data available from three studies—the Religious Order Study and the Rush Memory and Aging Project (ROS/MAP), the Mount Sinai Brain Bank cohort (MSBB), and the Mayo clinic RNA-seq study-covering 7 brain regions (cerebellum, temporal cortex, dorsolateral prefrontal cortex, parahippocampal gyrus, frontal pole, inferior frontal gyrus and superior temporal gyrus), as well as a published meta-analysis of these datasets was used to identify genes in the SM pathway that are differentially expressed in AD cases compared to controls. Gene expression changes were considered significant at an FDR-corrected p-value≤0.05.

### Metabolic Networks of Brain Regions

Genome-scale metabolic networks of brain regions were reconstructed and integrated with previously described post-mortem brain transcriptome data. Using iMAT algorithm, context-specific personalized metabolic networks were generated for each post-mortem sample in the dataset. Human cells in general do not proliferate rapidly and they tend to maintain their metabolic functions. Therefore, the biomass maintenance reaction, glutamate and glutamine exchange were chosen as the objective function for the brain regions. The dorsolateral prefrontal cortex samples were used for the analysis. Flux variability analysis (FVA) was performed to evaluate minimum and maximum flux for each reaction in the metabolic networks. If the minimum and maximum flux was 0, then the reactions were considered to be non-active and were assigned a state of 0, while the remaining reactions were considered to be active and assigned a state of 1. Analysis for all context-specific metabolic networks was carried out and a matrix of binary values was generated for all reactions in the context-specific metabolic networks. Reactions that were part of sphingolipid metabolism were selected using the subsystem definition. Fisher's exact test was used on the binarized values of reactions to identify reactions with p-value of <0.05 in AD versus CN samples. These reactions were identified as significant reactions in the groups. COBRA toolbox v3.044 was used for metabolic analysis that was implemented in MATLAB R2018a and academic licenses of Gurobi Optimizer v7.5 and IBM CPLEX v12.7.1 were used to solve LP and MILP problems.

### Neuroimaging Processing and Analysis

Participants of the Alzheimer's Disease Neuroimaging Initiative (ADNI) were used in the disclosed analyses. Demographic information, imaging scan data, neuropsychological test scores, and clinical information were downloaded from the ADNI data repository. As described in detail in previous studies, T1-weighted structural magnetic resonance imaging (MRI) scans were processed by using a widely employed automated MRI analysis technique (FreeSurfer) to extract cortical thickness. Pre-processed [¹³F] FDG positron emission tomography (PET) scans were downloaded. Methods for acquisition and processing of PET scans were described previously. [¹⁸F] FDG PET scans were intensity-normalized using a pons region of interest to create standardized uptake value ratio (SUVR) images. For surface-based whole brain analysis of cortical thickness on a vertex-by-vertex basis, the SurfStat software package was used to perform a multivariable analysis of generalized linear regression to examine the association of genetic variation on brain structural changes. Age, sex, years of education, intracranial volume, and magnetic field strength were used as covariates. In order to adjust for multiple comparisons, the random field theory correction method was used with p<0.05 adjusted as the level for significance. For whole brain analysis of brain glucose metabolism on a voxel-wise basis using the processed FDG PET images, SPM12 was used to investigate the effect of genetic variation on brain glucose metabolism across the whole brain. Age and sex were used as covariates. In order to adjust for multiple comparisons, the significant statistical parameters were selected to correspond to a threshold of p-value<0.05 (FDR-corrected).

### Assessment of SM Ratios Using Targeted Metabolomics in ADNI-1

For the investigation of SM ratios measured by targeted metabolomics using the Biocrates P180 kit, cohort data and statistical models that were previously described were used. For selection of the most informative SM ratio, all ratios between short-chain (chain length <C20) and long-chain (≥C20) SMs on metabolite levels were first calculated, not adjusted for medication. For each ratio, significant medications were then identified using backward selection based on the Bayesian Information Criterion. Significant medications were included as additional covariates extending the base models described previously for phenotype associations. Using the P_{gain} criterion, which is defined by the ratio of the minimum association p-value of the constituents of a ratio with the association p-value of the ratio and provides a measure of significance added by the ratio, the ratio of SM (d34:1) and SM (d43:1) was obtained, which had the largest overall P_{gain}.

### Replication Analysis of SM Ratios Using Targeted Lipidomics in ADNI-1

A more detailed lipidomics method was applied in the ADNI-1 samples to obtain better coverage of the sphingolipidome. In brief, extracted samples were run using reverse phase liquid chromatography coupled with a triple quadrupole mass spectrometer (Agilent 6490, Agilent). Characterization of sphingolipid isomers have been previously reported where repeated pooled runs using differing mass spectrometry conditions are used to obtain structurally informative fragments in MS/MS. Ratios were generated using 112 sphingolipid species and log 2-transformed. Linear regression with ADAS-Cog was performed with age, sex, BMI, HDL-C, total cholesterol, clinical triglycerides, fasting status and APOE e4 genotype as covariates. p-values were corrected for multiple correction comparison using the Benjamini and Hochberg approach.

Candidate mGWAS Analysis in ADNI-1

Genome-wide genotype data was downloaded for ADNI-1 participants from LONI. Genotype quality control (QC) included exclusion of samples and genotypes with <95% call rate and exclusion of variants that violated a Hardy-Weinberg-Equilibrium (HWE) test p-value of 1×10⁻⁵ or had a minor allele frequency (MAF)<5%. Autosomal mGWAS analysis was then performed with the three SMs (SM (d32:0), SM d(34:1), SM (d38:3)) previously reported as significantly associated with markers of AD, as well as the SM (d43:1)/SM (d34:1) ratio reported herein. As covariates, age, sex, diagnostic group, as well as the first five components derived by multidimensional scaling (MDS) analysis were included to account for population stratification. The threshold for genome-wide significance adjusted for four metabolic traits was p-value 51.25×10⁻⁸. Genetic associations were calculated using PLINK v1.949.

Phenotype GWAS and Global SM mGWAS Analysis in ADNI-1/GO/2

Genome-wide genotyping data of ADNI-1/GO/2 participants were collected using the Illumina Human 610-Quad, HumanOmni Express, and HumanOmni 2.5M BeadChips. Before imputation, standard QC procedures of GWAS data for genetic markers and subjects were performed (variant call rate <95%, HWE test p-value <1×10⁻⁶, and MAF<1%, participant call rate <95%, sex check and identity check for related relatives). Then, non-Hispanic Caucasian participants were selected using HapMap 3 genotype data and MDS analysis. Genotype imputation was performed for each genotyping platform separately using the Haplotype Reference Consortium (HRC) reference Panel r1.1 and merged afterwards, resulting in data on 1,576 individuals and 20,779,509 variants. Using this dataset, GWAS analyses were run for each outcome (A-T-N-C measures, clinical diagnosis, and metabolite levels) that included outcome-specific sets of covariates.

### Annotation of Genetic Variants and Gene-Wide Significance Thresholds

Previously reported metabolite associations for genes in the SM pathway were retrieved from SNiPA31, which was also used to identify overlapping expression quantitative trait loci (eQTLs) from multiple sources. Effect directions of genotype-metabolite and eQTL associations were obtained from previous studies. SNiPA was also used to project genetic variants to genes, a process that includes mapping of variants to genes via genomic location, links to genes via expression and protein QTLs, as well as location in a gene-associated promoter or enhancer region. The number of all genetic variants projected to a particular gene was used to derive gene-wise Bonferroni thresholds for significant genetic associations (p-value 50.05/(number of variants)). Furthermore, as SNPs within genes are correlated due to linkage disequilibrium and Bonferroni correction is often too conservative, permutation test was used, which provides a gene-based empirical p-value that corrects for the number of SNPs within each gene by randomly permuting the phenotypes multiple times (20,000 times) and performing statistical tests for all permuted data sets.

### In Vivo Mouse Models

APPswe/PS1dE9 (referred to as APP/PS1) and C57BI/6J (referred to as WT) mice were purchased from The Jackson Laboratory. The APP/PS1 is a double transgenic mouse expressing a chimeric mouse/human amyloid precursor protein (Mo/HuAPP695swe) and a mutant human presenilin 1 (PS1-dE9), both directed to CNS neurons. 50% of animals used in all experiments were male.

### Fingolimod Administration

To determine if fingolimod (e.g., Gilenya®, Novartis) oral administration achieves appropriate plasma concentration, 8 WT mice at 7 months old (50% females) were treated with fingolimod at 1 mg/kg/day for 4 wks. Plasma samples were collected at two time points (2^{nd} and 4^{th} weeks) after treatment and analyzed by UHPLC and MS-MS. Fingolimod levels in plasma were in ng/mL: 2^{nd} week=8.03±0.24 and 4^{th} week=10.02±0.4. The results indicate that oral administration is an appropriate route for mice experiments.

APP/PS1 mice and their WT littermates were used to examine fingolimod effects in vivo. Fingolimod treatment was provided in drinking water in a dark container and changed every 48 h to provide 1 mg/kg/day.

In vitro Electrophysiological Recordings

Mice were anesthetized with ketamine/xylazine (100/10 mg/kg) and decapitated with an animal guillotine. Horizontal hippocampal slices (400 μm) were prepared using a Vibratome slicer (VT 1000S; Leica) in ice-cold cutting solution containing the following: 130 mM potassium gluconate, 5 mM KCl, 20 mM HEPES acid, 25 mM glucose, 0.05 mM kynurenic acid, 0.05 mM EGTA-K, and pH equilibrated to 7.4 with KOH. After slicing, the tissue was allowed to recover for an hour before the beginning of experiments in artificial CSF (aCSF) that contained the following: 157 mM Na⁺, 136 mM Cl⁻, 2.5 mM K⁺, 1.6 mM Mg²⁺, 2 mM Ca²⁺, 26 mM HCO₃⁻, and 11 mM D-glucose.

LTP recordings were performed in an interface chamber (Fine Scientific Tools, Vancouver Canada) and slices were perfused with aCSF continuously bubbled with 95% O₂/5% CO₂, to maintain pH near 7.4 and the temperature was set at 34° C. Field excitatory post-synaptic potentials (fEPSPs) were recorded in the CA1 stratum radiatum with a glass electrode filled with aCSF (2-3 MΩ resistance). The fEPSPs were elicited by stimulating the Schaffer collateral fibers with a bipolar electrode. Input-output curves were obtained, and a stimulus that evoked ˜40% of the maximum fEPSP was selected to record the baseline. Baseline responses were obtained (15 min with an inter-stimulus interval of 20 s) before high-frequency stimulation (HFS) (one train of 100 stimuli at 100 Hz) was used to induce synaptic LTP. Responses were recorded for 60 min after HFS. The tungsten stimulating electrodes were connected to a stimulus isolation unit (Grass S88), and the recordings were made using an Axoclamp 2B amplifier (Molecular Devices) and then filtered (0.1 Hz to 10 kHz using −6 dB/octave). The voltage signals were digitized and stored on a PC using a DigiData 1200 A (Molecular Devices) for off-line analysis. The fEPSP slope was measured and expressed as a percentage of baseline. The data were analyzed using Axon™ pCLAMP™ software, and the results are expressed as the mean±standard error of the mean (SEM). Data were analyzed statistically using repeated measures ANOVA with the SPSS package.

### Novel Object Recognition (NOR)

Mice were habituated to experimental apparatus consisting of a gray rectangular open field (60 cm×50 cm×26 cm) for 5 min in the absence of any objects for 3 days. On the third day, after the habituation trial, mice were placed in the experimental apparatus in the presence of two identical objects and allowed to explore them for 5 min. After a retention interval of 24 h, mice were placed again in the apparatus, where one of the objects was replaced by a novel object. All sessions were recorded using Noldus Media Recorder software. Exploration of the objects was defined as the mice facing and sniffing the objects within 2 cm distance and/or touching them, assessed with ANY-maze software. The ability of the mouse to recognize the novel object (discrimination index) was determined by dividing the mean time exploring the novel object by the mean of the total time exploring the novel and familiar objects during the test session.

### Barnes Maze

The behavioral apparatus consisted of a white flat, circular disk with 20 holes around its perimeter. One hole held the entrance to a darkened escape box not visible from the surface of the board, allowing the subject to exit the maze. The escape chamber position remained fixed during all trials. Mice learn the location of the escape hole using spatial reference points that were fixed in relation to the maze (extra-maze cues). The task consisted of one habituation trial on day 1 where the escape hole was presented to the animal, the animal remained in the escape box for 2 min. After the habituation trial the training phase consisted of four 3-min trials of spatial acquisition for 4 consecutive days with a 15 min inter-trial interval. On the fifth day (probe trial) the escape box was removed, and the animals were allowed to explore the maze for 90 s. All sessions were recorded using Debut video software and assessed through ANY-maze software. For each trial, several parameters were recorded to assess performance. These include: the latency to locate the escape box, the number of incorrect holes checked prior to entering the escape box, as well as the distance traveled prior to locating the escape box. For the probe trial, time spent in the target quadrant and target hole were analyzed.

### Example 2

### Post-Mortem Brain RNA-Seq Data Demonstrates Global Transcriptomic Dysregulation of the SM Pathway in AD

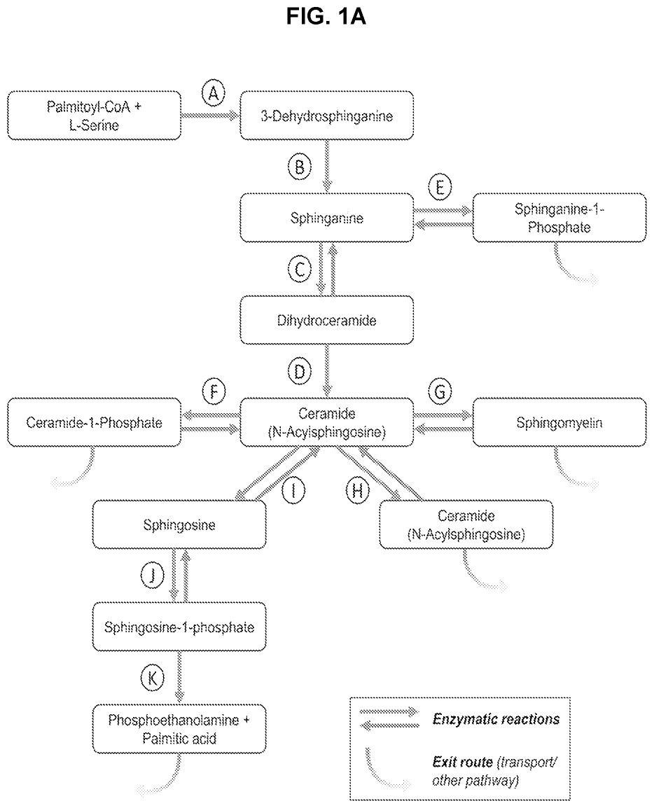

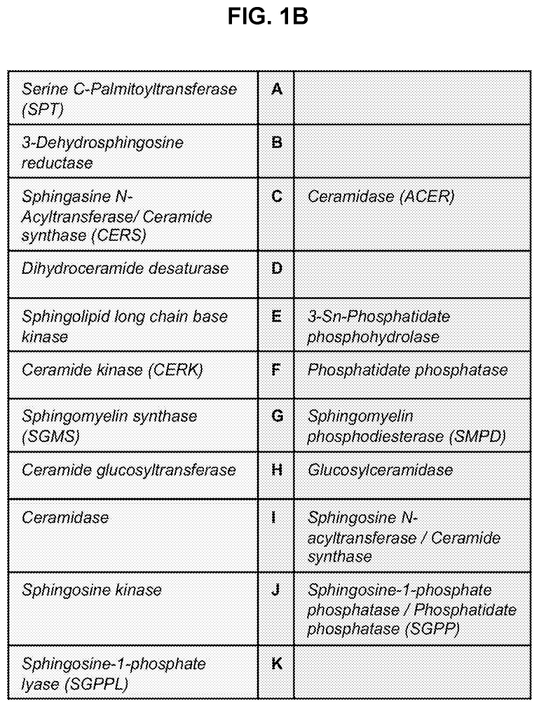

Gene expression changes of well-characterized enzymes in the sphingolipid pathway were analyzed from post-mortem brain RNA-seq data generated on seven brain regions (cerebellum, temporal cortex, dorsolateral prefrontal cortex, parahippocampal gyrus, frontal pole, inferior frontal gyrus, and superior temporal gyrus) in three independent cohorts (ROS/MAP, Mayo, and Mount Sinai) of 2114 brain samples, as well as the cross-region, cross-study meta-analysis. For this study, manual curation was performed for the sphingolipid subsystem definition of the human genome-scale metabolic reconstruction, resulting in the identification of a set of 35 enzymes catalyzing 18 enzymatic reactions within the SM pathway (FIG. 1). The reactions cover Cer and SM biosynthesis, as well as four exit routes, through sphinganine-1-phosphate, ceramide-1-phosphate, sphingomyelin, glycosphingolipids, and sphingosine-1-phosphate. Gene expression data were available for 31 of the 35 genes, the exceptions being CERS3, ACER1, ASAH2 and ENPP7. Low and/or no expression of these genes in the brain was confirmed in the GTEx Portal.

Analysis of differential gene expression showed significant (FDR-corrected) gene expression changes in brain tissue of AD cases vs. controls for 20 of the genes. Of those 20 genes, 19 showed differential expression in one or more studies/brain regions. Fourteen of these genes were also detected in the meta-analysis. Transcripts of SPTLC3 were not measured in all brain regions, hence it was not reported in the meta-analysis. DEGS1, on the other hand, was insignificantly but consistently upregulated in the single studies, leading to a detectable significant overall upregulation in the meta-analysis. Almost all of the genes showed significantly higher expression in AD cases, consistent across all brain regions. The exceptions were CERS5 (lower levels in cerebellum of AD cases; not significant in the meta-analysis), CERS6 (higher levels in cerebellum vs. lower levels in the parahippocampal gyrus of AD cases; not significant in the meta-analysis), and SMPD3 (lower levels in temporal cortex of AD cases; also significant in the meta-analysis).

### Example 3

Reconstructed Metabolic Networks Predict Differential Flux in AD for SM- and Cer-Associated Reactions Catalyzed by Enzymes with Elevated Gene Expression

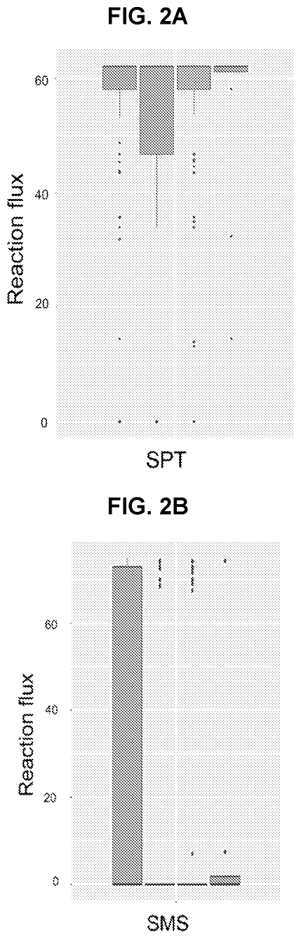

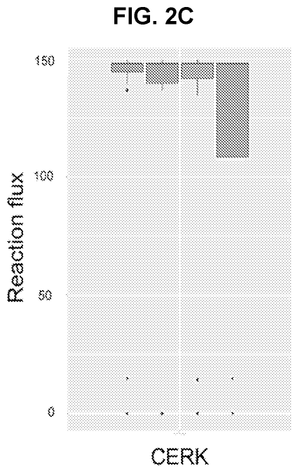

Brain region-specific metabolic reconstructions were used and integrated with the post-mortem brain RNA-seq data to identify reactions that had differential fluxes in AD vs. cognitively normal (CN) or control individuals. For the dorsolateral prefrontal cortex, reactions catalyzed by serine palmitoyltransferase (SPT, encoded by SPTLC1/2/3, enzyme A in FIG. 1), sphingomyelin synthase (SMS, encoded by SGMS1/2, enzyme G in FIG. 1), and ceramide kinase (CERK, encoded by CERK, enzyme F in FIG. 1) were identified as having significant flux differences, as shown in FIG. 2A-C. SPT catalyzes the first step in the biosynthesis of sphingolipids by condensing serine and palmitoyl-CoA to form 3-ketosphinganine, which is the rate-limiting step in the synthesis of SMs (FIG. 1). For this reaction, significant differences in flux values were found comparing AD and mild cognitive impairment (MCI) cases (FIG. 2A). Sphingomyelin synthase synthesizes sphingomyelin from ceramide. Here, AD transcriptomes having higher reaction fluxes compared to the CN samples were observed (FIG. 2B). Flux differences were further identified for the reaction catalyzed by ceramide kinase (CERK; phosphorylation of ceramide to form ceramide-1-phosphate) in AD and CN samples (FIG. 2C).

### Example 4

Genetic Association Studies with Markers of AD and Multimodal Neuroimaging Analysis Link the SM Pathway to AD Pathogenesis

Using gene-based association analysis in 1,576 participants of the AD Neuroimaging Initiative (ADNI) phases 1, GO and 2, genetic variants in the coding regions linked to seven of the 35 genes in the SM pathway were identified to be significantly associated with AD and its (bio)markers, which covered the whole spectrum of Amyloid, Tau, Neurodegeneration, Cognition (A-T-N-C) measures. A-T-N-C measures of AD are calculated by investigating genetic associations of CSF biomarker levels, brain atrophy (magnetic resonance imaging, MRI), brain glucose metabolism ([¹⁸F]FDG PET), cognition, and clinical diagnosis. In this analysis, Bonferroni-significance was determined by gene-specific thresholds correcting for the number of all genetic variants assigned to a certain gene. Associated markers included CSF Aβ₁₋₄₂ (CERS2, enzyme C in FIG. 1), the ratio between CSF tau (both total tau and p-tau) and CSF Aβ₁₋₄₂ (ACER2 (enzyme C in FIG. 1), PLPP2), region of interest-based measures of [¹⁸F] fluorodeoxyglucose positron emission tomography (FDG-PET; CERS3, SPHK2), cognitive performance measured, among others, by the 13-item cognitive subscale of the AD assessment scale (ADAS-Cog.13; CERS6, DEGS1), and clinical AD (CERS3, CERS6, DEGS1). Furthermore, a detailed whole brain analysis of brain glucose metabolism (FDG PET) on voxel-wise levels showed that rs1847325 in CERS3 (enzyme C in FIG. 1) and rs281380 in SPHK2 (enzyme J in FIG. 1) were significantly associated with increased brain glucose metabolism in the bilateral frontal, parietal, and temporal lobes (colored regions with corrected p-value <0.05). Previously, a study on clinico-pathologic AD dementia yielded an association with SMPD2 (enzyme G in FIG. 1) that is Bonferroni-significant at the gene-wide level.

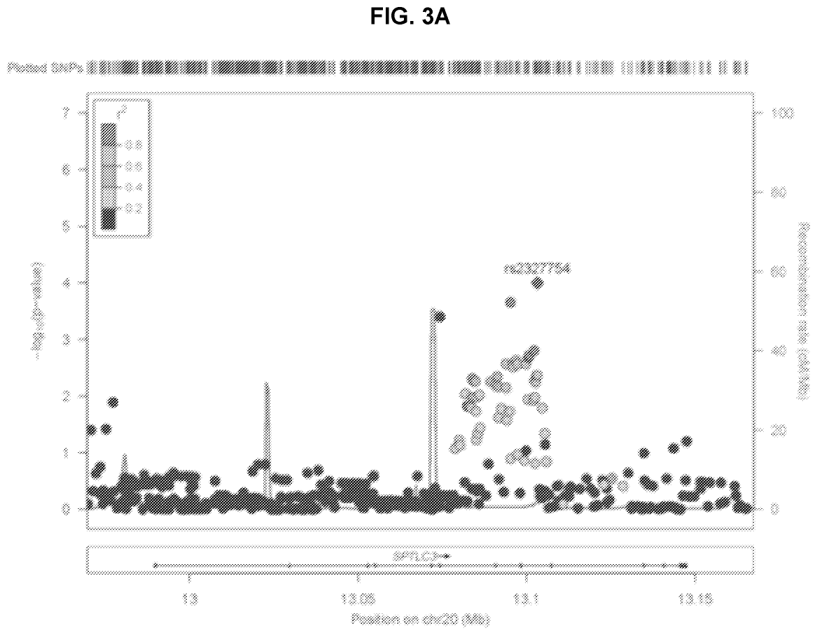

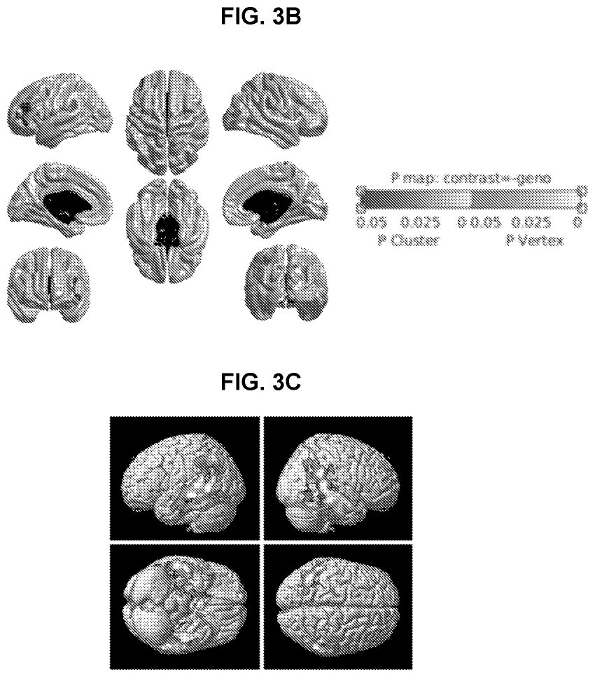

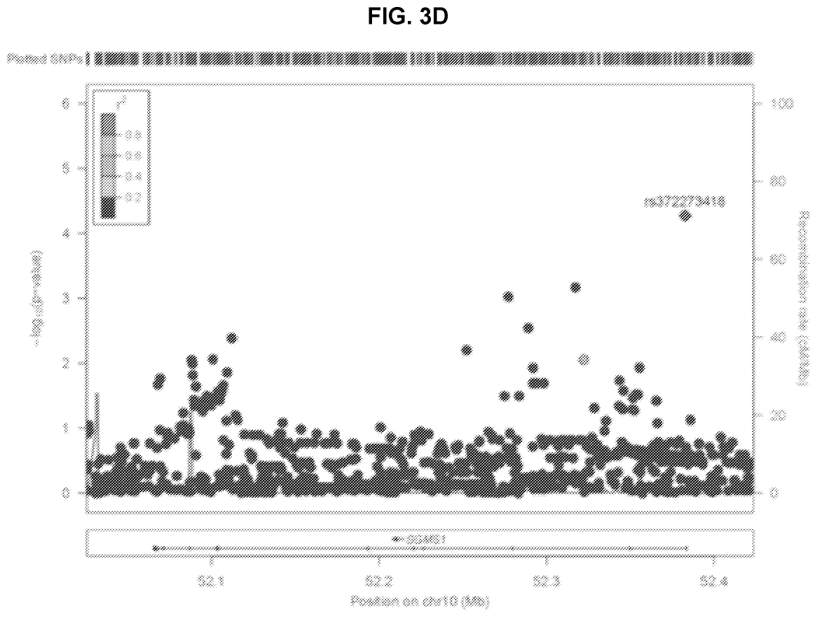

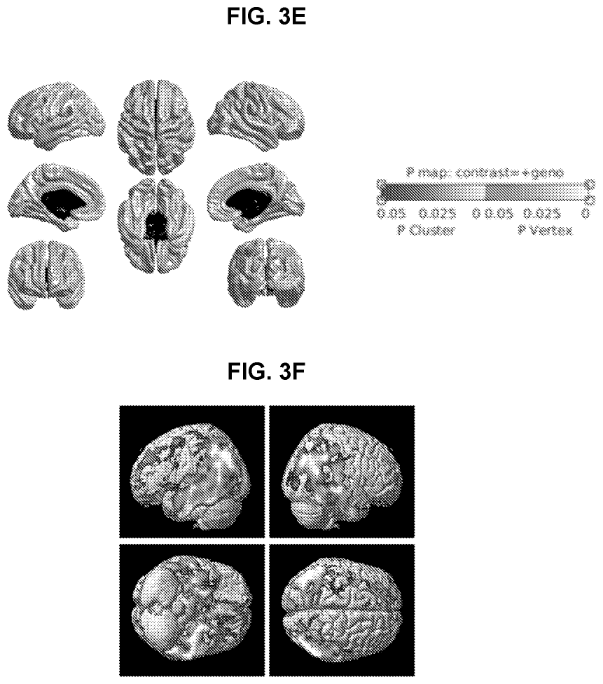

A less stringent p-value cutoff (adjusting for multiple testing by permutation as SNPs are correlated due to linkage disequilibrium) identified variants in two additional genes, SPTLC3 (enzyme A in FIG. 1) and SGMS1 (enzyme G in FIG. 1). SPTLC3 was associated with cognitive performance (corrected p-value=0.02; FIG. 3A), brain atrophy in focal regions of the bilateral temporal and frontal lobes (determined by detailed surface-based whole-brain analysis of cortical thickness measured from MRI scans on a vertex-wise level; colored regions with corrected p-value <0.05; FIG. 3B) and FDG-PET measures in the bilateral temporal and parietal lobes (colored regions with corrected p-value <0.05; FIG. 3C). SGMS1 was associated with brain glucose metabolism measured by region of interest-based FDG-PET (corrected p-value=0.02; FIG. 3D) that was mapped by whole brain analysis to the bilateral temporal, parietal, and frontal lobes, as well as the hippocampus (colored regions with corrected p-value <0.05; FIG. 3F). In addition, surface-based whole-brain association analysis showed a significant association with cortical thickness in the bilateral temporal, parietal, and frontal lobes, with the strongest association located in the entorhinal cortex (colored regions with corrected p-value <0.05; FIG. 3E).

### Example 5

### Expanded Plasma Metabolomics and Lipidomics Analysis Identifies the SM (d43:1)/SM (d34:1) Ratio as a Strong Intermediate Trait for Sphingolipid Dysregulation in AD

Sphingomyelin species (SMs) of differing lengths have been implicated in early vs. late stages of AD. SM (d34:1) is associated with CSF Aβ₁₄₂ pathology, while SMs with longer fatty acid chains (≥C20) are correlated with brain atrophy and cognitive decline. Utilizing the concept of metabolite ratios, which enables both removal of potentially remaining technical variance and modeling of enzymatic/pathway activity, ratios of shorter chain SMs (<C20) and longer chain SMs (≥C20) were selectively screened in the ADNI-1 dataset (n=732). This revealed the ratio of SM (d34:1) and SM (d43:1) as the metabolic trait most significantly associated with diagnosis of clinical AD (p-value=1.70×10⁻⁴, P_{gain}=178), brain atrophy in regions implicated in AD30 (p-value=7.64×10⁻⁶, P_{gain}=687.57) as well as cognition (measured by ADAS-Cog 13; p-value=4.36×10⁻⁶, P_{gain}=2544). The modified Alzheimer's Disease Assessment Scale cognitive subscale (ADAS-Cog 13-item scale) has all the original ADAS-Cog items with additional items that was aimed to increase the number of cognitive domains and range of symptom severity.

To expand upon and further validate this finding, the same cohort was examined (ADN11) using a more comprehensive lipidomics method covering a broader range of sphingolipids. In total, 112 sphingolipids were examined in serum samples (n=754), where chromatography enabled separation of some isomeric and isobaric species. Regression analysis (adjusting for age, sex, BMI, HDL-C, total cholesterol, triglycerides, APOE e4 and fasting status) between individual lipid species and lipid ratios (112 individual species, totaling 12,544 ratios) with ADAS-Cog 13 identified 3385 ratios associated with an uncorrected p-value of <0.05 and 1552 significant post FDR correction. This analysis confirmed that ratios of short to longer chain sphingomyelins, in particular the ratio of SM (d43:1)/SM (d34:1), presented with a positive association with ADAS-Cog 13 scores (FDR corrected p-value of 3.98×10⁻²).

### Example 6

### The SM (d43:1)/SM (d34:1) Ratio as an Intermediate for Genetic Screening and Locus Expansion Via Co-Associated Sms Identifies S1P Metabolism as a Potential Drug Target

To link SM readouts associated with AD to genes, metabolite genome-wide association studies (mGWAS) were performed with three SMs previously reported to be associated with markers of AD, as well as the selected ratio of SM (d43:1)/SM (d34:1). The discovery analysis was performed in a subset of 674 ADNI-1 participants that had genome-wide genotyping data available. While the three single SM species did not yield significant results, the SM ratio was associated with SPTLC3 (enzyme A in FIG. 1) at genome-wide significance corrected for 4 metabolic traits (lead SNP rs680379, p-value=1.01×10⁻⁹). This association replicated a previous finding in a larger population-based mGWAS investigating metabolite ratios (rs168622, r²=0.98 with rs680379, p-value=5.2×10⁻²⁵).

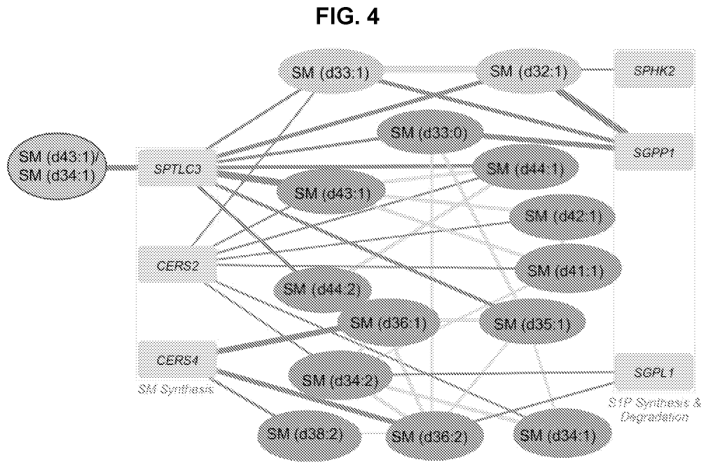

Investigation of the SPTLC3 (enzyme A in FIG. 1) locus using the large collection of metabolite-genotype associations in the SNiPA database) revealed significant links to several additional SM species. To obtain a comprehensive map of genetic influences on SM levels across the whole SM pathway, gene-based association analyses including all 35 genes in the pathway were used again analogously to the analysis of associations with markers of AD. To this end, an expanded set of 1,407 ADNI participants with SM readouts and genome-wide genotype information available were used, as well as two large population-based mGWAS studies that included SM levels. Genome-wide and gene-wide significant associations with a set of 14 related SMs for six genes were found (FIG. 4). Three of the encoded enzymes are involved in SM synthesis (SPTLC3, CERS2, CERS4), while the other three function in synthesis and degradation of S1P (SPHK2, SGPP1, SGPL1), a central exit route of the pathway. Notably, the significant associations include all three SMs that were previously identified (SM (d33:0), SM (d34:1), and SM (d38:2)), highlighting a potential role for S1P metabolism and signaling in AD pathogenesis.

### Example 7

Treatment of Amyloidogenic APP/PS1 Mice with Fingolimod Reveals Beneficial Effects of S1P Modulation on Behavioral and Synaptic Plasticity Deficits

To functionally investigate the involvement of deregulated S1P metabolism in amyloid pathology along with strategies to counter potential links to AD pathogenesis, a drug repositioning approach was applied by treating amyloidogenic APP/PS1 mice with fingolimod (FTY720), an FDA-approved drug for the use in the relapsing-remitting form of multiple sclerosis. The APP/PS1 mice are transgenic mice expressing chimeric amyloid precursor protein (APP) and mutant human presenilin 1 (PS1) and are valuable models for studying AD progression and the effects of drugs on AD. The immunomodulating compound is a sphingosine analog that, after endogenous phosphorylation by sphingosine kinases 1 and 2, broadly binds to S1P receptors (S1PR1/3/4/5).

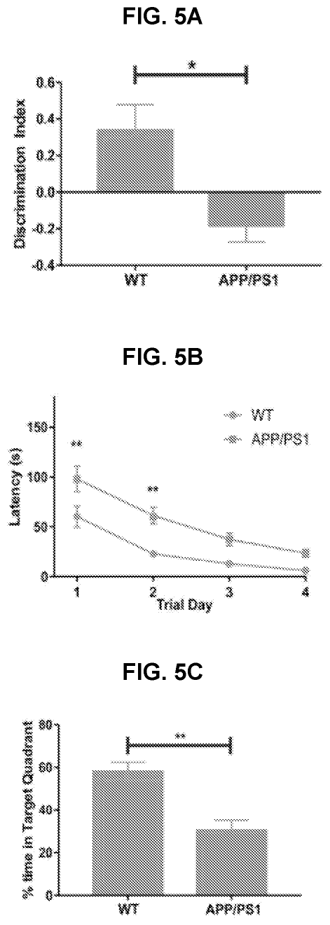

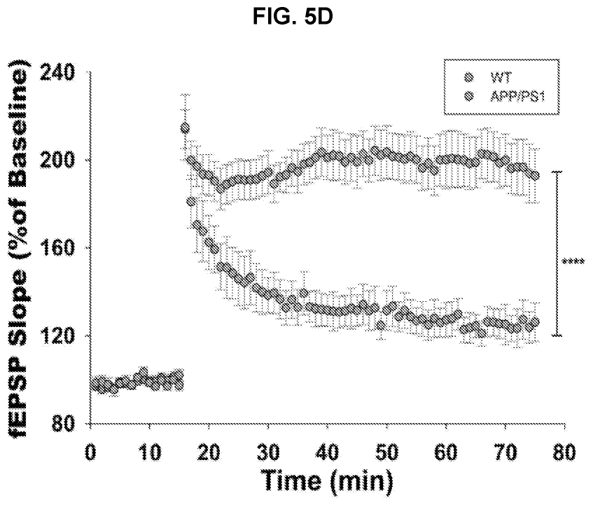

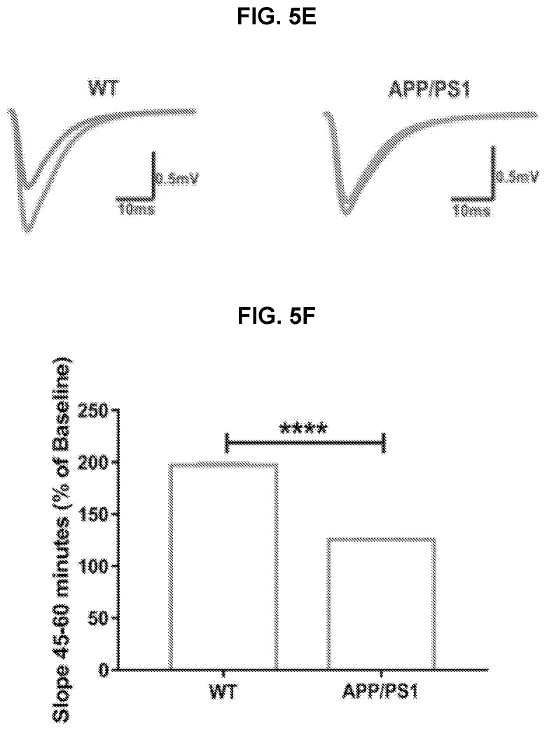

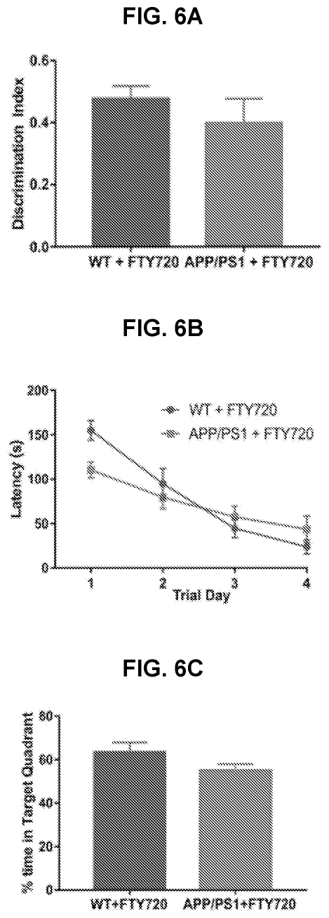

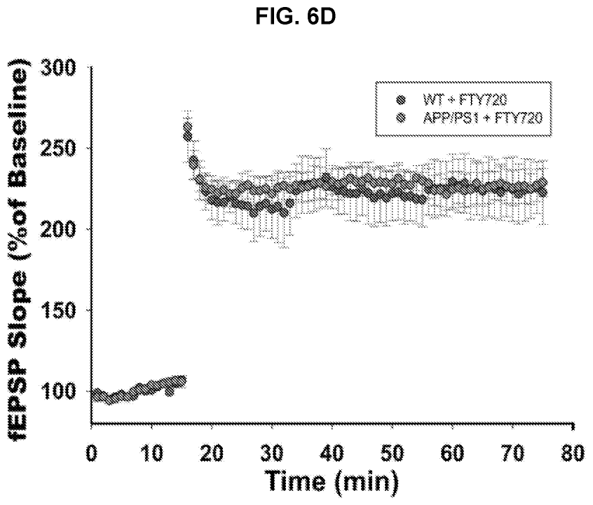

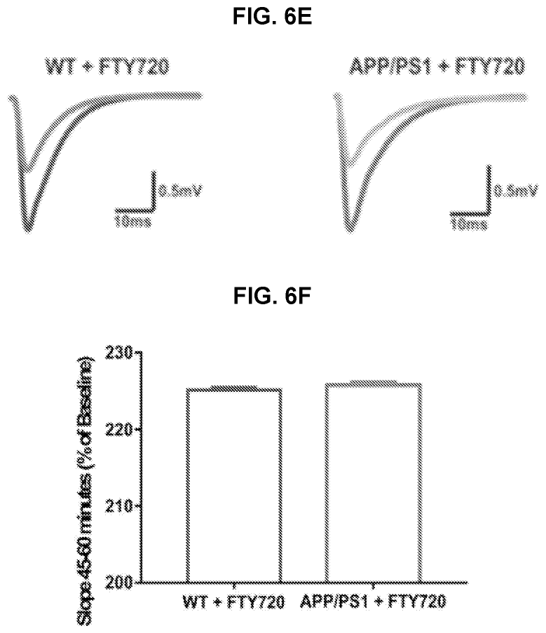

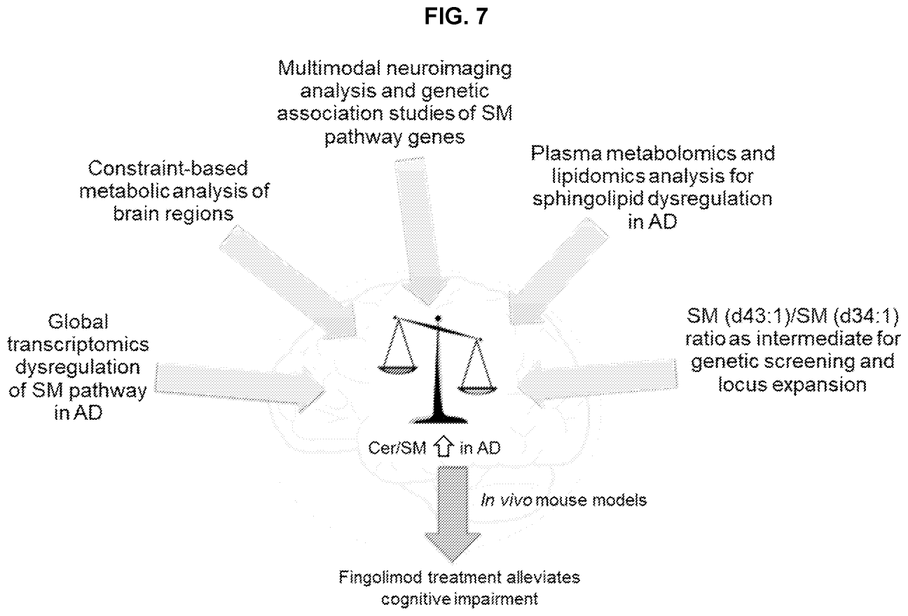

To corroborate previous studies with similar ages of mice, APP/PS1 mice (n=6, 50% female) and WT mice (n=6, 50% female) were phenotyped at 7 months old (m.o.) using a battery of behavioral tests. The novel object recognition (NOR) test and Barnes Maze task were utilized to assess episodic and spatial memory, respectively. After behavioral testing, synaptic transmission was evaluated using electrophysiology experiments at the Schaffer collateral-CA1 synapse. APP/PS1 mice had a significant deficit in the NOR test (Discrimination index (DI)=−0.18±0.18, FIG. 5A) compared to WT mice (DI=0.33±0.24; t₅=3.25; p-value=0.02). During the Barnes Maze task, APP/PS1 mice showed a mild deficit in spatial learning abilities (t₃₆=3.098; p-value=7.5×10⁻³ on 1^{st} day and t₃₆=3.156; p-value=7.5×10⁻³ on 2^{nd} day) (FIG. 5B) and memory retention by the less time spent in the target quadrant (30.39±4.8 vs. 57.97±4.4, t₈=3.939; p-value=4.3×10⁻³) (FIG. 5C) compared to their WT littermates. Furthermore, they showed abnormal long-term potentiation (LTP) both at the early and maintenance phases in the CA3-CA1 synapse (FIG. 5D-F), where APP/PS1 mice could not maintain the potentiation 40 min after HFS (112.7%±1.8) compared to WT mice (188.4% 3.06; F_{(1,38)}=95.37, p-value <0.0001). Of note, basal synaptic transmission as evaluated by the input-output relationship was similar in the two groups.

In a second experiment, mice were treated with fingolimod (1 mg/kg/day) for eight weeks (APP/PS1, n=6, 50% female; WT, n=6, 50% female; all mice 7 m.o. at the beginning of treatment). It was found that fingolimod-treated APP/PS1 mice had similar values in NOR-DI as compared to treated WT mice (0.40±0.13 vs. 0.47±0.08; t₈=0.95; p-value=0.38) (FIG. 5G). Comparison of APP/PS1 treated vs. WT treated mice showed that the percentage of time spent in the target quadrant for treated APP/PS1 was similar to treated WT mice (54.81±9.15 vs. 63.46±11.51, t₁₄=1.677; p-value=0.12) (FIG. 5I). Latency across training days also showed no difference between groups (t₃=0.4697; p-value=0.67) (FIG. 5H). Remarkably, fingolimod treatment of APP/PS1 mice significantly augmented the normalized slope of fEPSP at the CA3 to CA1 synapse after HFS (FIGS. 5J and 5K). Comparison of the average slope percent change during the last 20 min of the maintenance phase also showed no difference (225.8%±1.86 vs. 225.1%±1.78; t₃₈=1.163; p=0.25) (FIG. 5L).

Next, APP/PS1 mice treated with fingolimod were compared to untreated APP/PS1 mice (9 m.o. 50% female, n=6 treated, n=4 untreated) and analysis of the latency during training showed a deficit in untreated compared to treated APP/PS1 mice (RM-ANOVA F=3.1; p=0.041). These data indicate that prolonged S1P pathway modulation can rescue both the proposed cellular mechanism of hippocampus related memory (synaptic LTP) and the cognitive deficits per se in amyloidogenic APP/PS1 mice.

In the described studies, the SM pathway, and multi-omics links to pathogenic processes in AD were systemically analyzed. The key findings from the multi-omics work include: (a) differentially expressed genes in the SM pathway of AD patients were identified by using post-mortem brain transcriptome data of 2114 samples; (b) comparison of 1708 context-specific metabolic reconstruction of the brain regions showed differences in the reaction fluxes for AD and CN samples; (c) multimodal neuroimaging analysis of 1576 individuals identified genetic variants linked to genes in the SM pathway and associated with AD pathogenesis; (d) plasma metabolomic and lipidomic analysis identified the SM (d43:1)/SM (d34:1) ratio as a strong intermediate trait for sphingolipid dysregulation in AD; (e) metabolite genome-wide association studies (mGWAS) identified S1P metabolite as a potential AD drug target; and (f) experimental analyses of amyloidogenic APP/PS1 mice treated with fingolimod revealed beneficial effects of S1P modulation and alleviated cognitive impairment in mice.

It was demonstrated that, on the gene expression level, the SM pathway is globally dysregulated across brain regions in samples of AD cases compared to controls. It was found that 20 out of 35 genes encoding the core enzymes in the pathway are significantly differentially expressed in the AD population. The only sub-pathway that appears to be unaffected by or uninvolved in the disease is the synthesis and recycling of glycosphingolipids. Using constraint-based metabolic networks of brain regions integrated with post-mortem brain transcriptome data, it was shown that the differential expression of the enzymes involved in at least three reactions are predicted to result in significant flux differences in AD cases versus controls. While flux differences cannot be directly interpreted with respect to the resulting metabolic changes, there is ample evidence from metabolomics studies that the pathway exhibits differential output in AD.

The association of genes in the SM pathway with A-T-N-C measures of AD was assessed by investigating genetic associations of CSF biomarker levels, brain atrophy (MRI), brain glucose metabolism ([¹⁸F] FDG PET), cognition, and clinical diagnosis. Ten of the 35 genes in the pathway showed significant associations with at least one (endo)phenotype at the gene level. Although not genome-wide significant, this large coverage of genes in the SM pathway suggests that there might be at least a small fraction of genetic risk predisposition to AD attributable to the pathway as a whole. Using SM levels as intermediate traits for genetic association, screening further revealed six central enzymes in the pathway to be genetically influencing levels of a network of 14 SM species. As all of the genetic variants associated with SM levels were linked to the respective enzymes via expression quantitative trait loci, this indicates that some of the genetic links between the pathway and markers of AD may be mediated by altered regulation of SM levels via genetically influenced differential gene regulation.

While associations from the analysis of differential gene expression in brain tissue as well as from the phenotype GWASs were broad and generally implicated SM pathway function, the associations from the SM mGWASs linked two central pathway routes: global SM synthesis and S1P metabolism. Based on previous mGWAS analysis, genetic associations with core enzymes involved in the primary synthesis of SM metabolites are expected. However, the specific association with one particular exit route out of the pathway (via sphingosine and S1P) is striking. Five of the six detected genes (SPTLC3, CERS2, CERS4, SPHK2 and SGPL1) were also found to be significantly linked to AD either through differential gene expression or via genetic associations or both, which suggests that S1P metabolism may be relevant to disease.

S1P is known to be involved in endothelial barrier function in a context-dependent manner. Decreased S1P by lipopolysaccharide (LPS) treatment produced blood brain barrier (BBB) abnormalities, and increased activity of SGPP1 and S1PR. Chronic BBB leakiness is associated with cognitive impairment, but not with signs of brain inflammation. S1P in general increases neuronal and circuit excitability. Depletion of the S1P producing enzyme SPHK1 induces an impairment of mossy fiber—CA3 LTP and deficits in spatial reference memory. Depletion of SPHK2 produced lower levels of hippocampal S1P, reduced histone acetylation and deficits in spatial memory as well as impaired contextual fear extinction. Thus, S1P, SPHK1 and SPHK2 play specific roles in brain areas serving specific memory functions through intracellular S1P effects as well as signaling pathways downstream of S1P GPCRs. A recent study showed that Aβ₁₋₄₂ enhanced SPHK1 expression and activity after 24 h, but down-regulated them after 96 h and had no effect on SPHK2. Aβ₁₋₄₂ and SKI II induced free radical formation, disturbed the balance between pro- and anti-apoptotic proteins and evoked cell death in PC12 cells while SP1 rescued part of this damage. S1P may act as a second messenger, but it can also be transported to the extracellular space and may affect cell function via stimulation of the receptors (S1PR1-5). Two modulators of SP1 R1 (fingolimod and SEW2871) have been shown to improve Aβ mediated behavior abnormalities and decrease tau phosphorylation.

To explore the effect of drugs, APP/PS1 mice were used. Fingolimod is a sphingosine-1-phosphate receptor modulator approved for treatment of multiple sclerosis in the US. In absence of the drug treatment, it was observed that APP/PS1 mice had a significant deficit in the novel object recognition test (NOR) and other array of tests as compared to the WT mice. Upon administration of fingolimod, it was found that fingolimod-treated APP/PS1 mice had similar values in NOR-DI as compared to treated WT mice. These results suggested that fingolimod modulated S1P pathway and was able to alleviate cognitive deficits in APP/PS1 mice.

The approach provided herein opens the possibility of repurposing fingolimod, or other S1P modulators, for the treatment of AD. Fingolimod has been shown to modulate both amyloid and tau pathology in AD models and it has been proposed to be neuroprotective by modulating S1P signaling in the brain. Rescuing both memory (synaptic LTP) and the behavior itself (substrate and end-result) with fingolimod is a compelling finding, which provides evidence for dysregulated S1P signaling in AD mice and further supports the identification of this pathway as a high priority candidate AD drug target. The effect of fingolimod in APP/PS1 on behavior and synaptic transmission can be direct or through the activation of S1P receptors, or both, since they are not mutually exclusive. Fingolimod is likely one of a several compounds approved or being tested for other neurodegenerative diseases that can be repurposed. The described studies integrate diverse types of multi-omics data from AD patients and an animal model to identify multiple, dysregulated steps in SM metabolism. The studies provide a link between SM dysregulation and changes in brain function. Furthermore, the described studies suggest that repurposing drugs that target SM metabolic enzymes, such as the S1P receptor, could correct the dysregulation and potentially improve memory and synaptic function. Thus, using the disclosed multi-omics approach to analyze big data led to the understanding of the sphingolipid pathway and strategies for novel drug discovery in AD.

1-4. (canceled)

5. A method for stratifying and treating a subject having a neurological disorder, or at risk of developing a neurological disorder, based on the subject's metabolic profile, the method comprising:
analyzing a sample from a subject to determine concentration levels or ratios of one or more biomarker metabolites related to ceramide and sphingomyelin anabolism, catabolism, or homeostasis in the sample from the subject;
determining if the subject has a metabolic defect related to disrupted ceramide and sphingomyelin anabolism, catabolism, or homeostasis, or if the subject's gut microbiome has a defect related to disrupted ceramide and sphingomyelin anabolism, catabolism, or homeostasis, or combinations thereof based on the measured concentration levels and calculated ratios of the one or more ceramide and sphingomyelin anabolism, catabolism, or homeostasis biomarker metabolites in the sample as compared to a control sample;
stratifying the subject into a subgroup of subjects, wherein an individual subgroup of subjects is defined by a unique and specific ceramide and sphingomyelin anabolism, catabolism, or homeostasis profile based on the measured concentration levels and calculated ratios of the one or more biomarker metabolites in the sample as compared to a control sample and the biomarker metabolite defect determined for the subject.

6. The method of claim 5, further comprising treating the neurological disorder by administering to the subgroup of subjects an effective amount of a therapy sufficient to attenuate, reduce, or eliminate the symptoms of neurological disorder, wherein the therapy is determined by the unique and specific metabolic profile of the subgroup of subjects.

7. The method of claim 5, wherein the one or more biomarker metabolites comprises one or more of:
sphingomyelins (SM): SM (OH) C14:1 (SM C15:0); SM C16:0; SM (OH) C16:1 (SM C17:0); SM C16:1; SM C18:0; SM C18:1; SM C20:2; SM (OH) C22:1 (SM C23:0); SM (OH) C22:2 (SM C23:1); SM C24:0; SM (OH) C24:1 (SM C25:0); SM C24:1; SM C26:0; SM C26:1; SM (d43:1); SM (d34:1); SM (d32:0); SM (d38:3); SM (d33:0); SM (d38:2); SM (d33:1); SM (d32:1); SM (d44:1); SM (d42:1); SM (d41:1); SM (d35:1); SM (d36:1); SM (d44:2); SM (d34:2); SM (d36:2); or combinations thereof;
ceramides (Cer): Cer (d18:1,14:0); Cer (d18:1,16:1); Cer (d18:0,16:1); Cer (d16:1,16:0); Cer (d18:2,16:0); Cer (d18:1,16:0); Cer (d18:0,16:0); Cer (d16:0,18:1); Cer (d16:1,18:0); Cer (d18:1,18:0); Cer (d18:1, 22:1); Cer (d18:0, 22:1); Cer (d18:2, 22:0); Cer (d18:1, 22:0); Cer (d18:1, 23:1); Cer (d18:0, 23:1); Cer (d18:2, 23:0); Cer (d18:1, 23:0); Cer (d18:1, 24:2); Cer (d18:0, 24:2); Cer (d16:1, 24:1); Cer (d18:2, 24:1); Cer (d18:1, 24:1); Cer (d18:0, 24:1); Cer (d16:1, 24:0); Cer (d18:2, 24:0); Cer (d18:1, 24:0); Cer (d18:0, 24:0); Cer (d20:1, 24:0); Cer (d20:0, 24:0); Cer (d18:1, 26:1); Cer (d18:0, 26:1); Cer (d18:2, 26:0); Cer (d18:1, 26:0); Cer (d18:0, 26:0); or combinations thereof; or
sphingosine-1-phosphate (S1P).

8. (canceled)

9. The method of claim 5, wherein the one or more biomarker metabolites related to ceramide or sphingomyelin metabolism comprises one or more sphingomyelins (SM): SM (OH) C14:1 (SM C15:0); SM C16:0; SM (OH) C16:1 (SM C17:0); SM C16:1; SM C18:0; SM C18:1; SM C20:2; SM (OH) C22:1 (SM C23:0); SM (OH) C22:2 (SM C23:1); SM C24:0; SM (OH) C24:1 (SM C25:0); SM C24:1; SM C26:0; SM C26:1; SM (d43:1); SM (d34:1); SM (d32:0); SM (d38:3); SM (d33:0); SM (d38:2); SM (d33:1); SM (d32:1); SM (d44:1); SM (d42:1); SM (d41:1); SM (d35:1); SM (d36:1); SM (d44:2); SM (d34:2); SM (d36:2); or combinations thereof; and wherein one or more ratios of the SM is calculated.

10. The method of claim 9, wherein the one or more biomarker metabolites related to ceramide or sphingomyelin metabolism comprises SM (d43:1) and SM (d34:1), and wherein a ratio of SM (d43:1)/SM (d34:1) is calculated.

11. The method of claim 10, wherein the subject is determined as having the neurological disorder or an increased risk of the neurological disorder when the sample comprises a higher ratio of SM (d43:1)/SM (d34:1) compared to the control sample.

12. (canceled)

13. The method of claim 5, further comprising:
administering to the subject a therapeutically effective amount of one or more ceramides, sphingomyelins, and/or any pharmaceutically acceptable derivatives, esters, salts, solvates, hydrates, analogs, or prodrugs thereof; and/or
administering to the subject a therapeutically effective amount of one or more therapeutic agents capable of modulating (increasing or decreasing) the concentration levels or ratios of one or more primary or sphingomyelins, activating the endogenous production of one or more ceramides or sphingomyelins, and/or decreasing the breakdown of one or more ceramides or sphingomyelins; and/or
administering to the subject a therapeutically effective amount of a sphingosine-1-phosphate receptor (S1PR) modulator including one or more of fingolimod, SEW2871, siponimod, ozanimod, ceralifimod, GSK2018682, ponesimod, KRP203, cenerimod, amiselimod (MT-1303), etrasimod, laquinimod, derivatives thereof, or combinations thereof.

14. A method for detecting a neurological disorder in a subject, the method comprising:
analyzing a sample from a subject and a control sample from a control subject or population of subjects with normal cognition;
measuring the concentration levels or ratios of one or more biomarker metabolites related to ceramide or sphingomyelin metabolism in the sample from the subject and the control sample; and
determining the subject as having a neurological disorder or an increased risk of a neurological disorder when the concentration levels or ratios of the one or more biomarker metabolites in the sample from the subject are different from (greater than or less than) the concentration levels or ratios of the one or more biomarker metabolites in the control sample.

15-16. (canceled)

17. The method of claim 14, wherein the one or more biomarker metabolites comprises sphingosine-1-phosphate (S1P), and wherein the subject is determined as having the neurological disorder or an increased risk of the neurological disorder when the sample comprises lower concentration levels of S1P compared to the control sample.

18-21. (canceled)

22. The method of claim 14, wherein the one or more biomarker metabolites comprises one or more ceramides (Cer), and wherein the subject is determined as having the neurological disorder or an increased risk of the neurological disorder when the sample comprises higher concentration levels of one or more Cer or a higher Cer/sphingomyelin (SM) ratio compared to the control sample.

23. The method of claim 14, further comprising treating the subject with a therapeutically effective amount of a compound capable of modulating the concentration levels or ratios of the one or more biomarker metabolites related to ceramide or sphingomyelin metabolism to reduce the negative effects of the neurological disorder.

24. The method of claim 23, wherein the compound modulates the concentration levels or ratios of one or more sphingolipids in the subject; wherein the compound increases the concentration levels of sphingosine-1-phosphate (S1P), modulates signaling by S1P, or a combination thereof; wherein the compound modulates the ratio of sphingomyelin (SM) (d43:1)/SM (d34:1); and/or wherein the compound decreases the concentration levels of one or more ceramides (Cer) and/or the ratio of Cer/SM.

25-27. (canceled)

28. The method of claim 23, wherein the compound comprises a sphingosine-1-phosphate receptor (S1PR) modulator including one or more of fingolimod, SEW2871, siponimod, ozanimod, ceralifimod, GSK2018682, ponesimod, KRP203, cenerimod, amiselimod (MT-1303), etrasimod, laquinimod, derivatives thereof, or combinations thereof.

29-30. (canceled)

31. The method of claim 14, wherein the neurological disorder is a neuropsychiatric or neurodegenerative symptom associated with neurological diseases or cognitive impairment, including depression, anxiety, dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's disease, young-onset dementia, Korsakoff's syndrome, Huntington's disease, HIV-associated neurocognitive disorders, multiple sclerosis, or other cognitive impairment, neuropsychiatric, or neurodegenerative disorders.

32-41. (canceled)

42. A method for detecting a neurological disorder in a subject, the method comprising:
analyzing a sample from a subject and a control sample from a control subject or population of subjects with normal cognition;
measuring the concentration levels of one or more enzymes related to ceramide or sphingomyelin metabolism in the sample from the subject and the control sample; and
determining the subject as having a neurological disorder or an increased risk of a neurological disorder when the concentration levels of the one or more enzymes in the sample from the subject are different from (greater than or less than) the concentration levels of the one or more enzymes in the control sample.

43. The method of claim 42, wherein the one or more enzymes related to ceramide or sphingomyelin metabolism comprises one or more of serine palmitoyltransferase (SPTLC1; SPTLC2; SPTLC3), sphingomyelin synthase (SGMS1; SGMS2), sphingomyelin phosphodiesterase (SMPD1; SMPD2), ceramide kinase (CERK), phosphatidate phosphatase (PLPP2), ceramidase (ASAH1), ceramide synthase (CERS2; CERS3; CERS4), sphingosine kinase (SPHK1; SPHK2), sphingosine-1-phosphate phosphatase (SGPP1), sphingosine-1-phosphate lyase (SGPL1), or combinations thereof.

44-45. (canceled)

46. The method of claim 42, further comprising performing neuroimaging analysis on the subject and the control subject or population of subjects with normal cognition, and correlating the results of the neuroimaging analysis with the measured concentration levels of the one or more enzymes related to ceramide or sphingomyelin metabolism in the sample from the subject and the control sample, to link the results of the neuroimaging analysis to the subject's metabolic profile: wherein the neuroimaging analysis assesses brain atrophy, brain glucose metabolism, or a combination thereof; and wherein the neuroimaging analysis comprises structural magnetic resonance imaging (MRI), molecular [¹⁸F] fluorodeoxyglucose (FDG) positron emission tomography (PET), or a combination thereof.

47-48. (canceled)

49. The method of claim 42, further comprising treating the subject with a therapeutically effective amount of a compound capable of modulating the concentration levels of the one or more enzymes related to ceramide or sphingomyelin metabolism to reduce the negative effects of the neurological disorder.

50. The method of claim 49, wherein the compound comprises a sphingosine-1-phosphate receptor (S1PR) modulator including one or more of fingolimod, SEW2871, siponimod, ozanimod, ceralifimod, GSK2018682, ponesimod, KRP203, cenerimod, amiselimod (MT-1303), etrasimod, laquinimod, derivatives thereof, or combinations thereof.

51. (canceled)

52. The method of claim 42, wherein the neurological disorder is a neuropsychiatric or neurodegenerative symptom associated with neurological diseases or cognitive impairment, including depression, anxiety, dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's disease, young-onset dementia, Korsakoff's syndrome, Huntington's disease, HIV-associated neurocognitive disorders, multiple sclerosis, or other cognitive impairment, neuropsychiatric, or neurodegenerative disorders.
